(19) United States
(12) Patent Application Publication
(10) Pub. No.: US 2009/0271123 A1
(43) Pub. Date: Oct. 29, 2009

(54) ORDERING GENES BY ANALYSIS OF EXPRESSION KINETICS

(76) Inventor: Uri ALON, Tel Aviv (IL)

Correspondence Address: Dr. D. Graeser Ltd.;c/o The Discovery Dispatch, 9003 Florin Way, Upper Marlboro, MD, 20772, US

(21) Appl. No.: 12/432,774

(22) Filed: Apr. 30, 2009

Related U.S. Application Data
(63) Continuation of application No. 10/514,233, filed on Nov. 19, 2004, now abandoned.
Application No. 10/514,233 is a continuation-in-part of application No. 10/161,604, filed on Jun. 5, 2002, now abandoned.

(51) Int. Cl. C12Q 1/68; G06F 17/10; G06F 19/00

(52) U.S. Cl. 702/19; 435/6; 703/2

(57) ABSTRACT

A method for analyzing the temporal behavior of gene expression for a group of genes which are part of a biological system or subsystem. Preferably, such an analysis enables the order of expression of such genes to be determined. More preferably, the temporal behavior of gene expression is assessed according to the analysis of the kinetics of gene transcription. According to a preferred embodiment of the present invention, the kinetics of gene transcription are measured according to promoter activity of a plurality of genes. More preferably, such kinetics are measured in a living organism or a portion of such an organism, such as a cell for example. For single-celled organisms, such as bacteria for example, the kinetics may easily be measured for the entirety of the living organism.

### FIELD OF THE INVENTION

The present invention is of a method for analyzing expression kinetics of genes, for ordering genes in a particular biological system or subsystem, and in particular, for such a method in which the temporal behavior of gene transcription is analyzed with regard to the biological function of the system.

### BACKGROUND OF THE INVENTION

Gene regulation networks are complex and represent the interaction of gene expression with the biological function of the products of that expression. In particular, proteins which interact as part of a biological system or subsystem may feature coordinate regulation of their respective genes, thereby enabling specific proteins to be produced in a particular order, for example. Such regulation clearly is required for the overall function of the organism. As a simplistic example, a first protein which upregulates (increases the activity of) a particular biological system or subsystem may not be produced when that system or subsystem is being downregulated (having its activity reduced).

One example of such a biological subsystem is the flagella of the bacterium E. coli. Under the proper conditions, the bacterium E. coli synthesizes multiple flagella, which allow it to swim rapidly. Classical genetics showed that the 14 flagella operons are arranged in a regulatory cascade of three classes (1-5) as shown in background art FIG. 1.

As shown in FIG. 1 (1, 2), the master regulator FlhDC turns on class 2 genes, one of which, FliA, turns on class 3 genes. A checkpoint ensures that class 3 genes are not turned on until basal body-hook structures (BBH) are completed. This is implemented by FlgM, which binds and inhibits FliA. When BBH are completed, they export FlgM out of the cell, leaving FliA free to activate the class 3 operons (9, 27, 28). It should be noted that flgM is transcribed from both a class 2 (flgAMN) and a class 3 (flgMN) promoter.

The class 1 operon encodes the transcriptional activator of class 2 operons. Class 2 genes include structural components of a rotary motor called the basal body-hook structure, as well as the transcriptional activator for class 3 operons. Class 3 includes flagellar filament structural genes and the chemotaxis signal transduction system that directs the cells' motion. A checkpoint mechanism ensures that class 3 genes are not transcribed before functional basal body-hook structures are completed.

FIG. 1 clearly illustrates the interdependence of biological function and the temporal behavior of gene expression, or the “timing” of gene transcription. Such interdependence is required in order for the bacterium to efficiently build the flagellum, or any other structure, which forms a biological subsystem. Furthermore, such interdependence may even extend to a plurality of subsystems or even an entire biological system, such as the bacterium itself. Yet, the timing of gene transcription has not been effectively analyzed for large sets of genes, nor has it been effectively analyzed for many smaller sets of genes. Indeed, many such smaller sets of genes, the existence of which may be expected on the basis of the requirements for biological functioning of an organism, have probably not been detected, let alone analyzed.

More generally, there is a great deal of interest in understanding the design principles underlying the structure and dynamics of gene regulation networks. Recent studies addressed the challenge of mapping the structure of transcriptional networks based on genomic data. These approaches aim to determine which transcription factors regulate which genes. However, determining the dynamic behavior of these systems requires specifying not only the network connectivity, but also the kinetic parameters for the various regulation reactions. Standard biochemical methods of measuring these kinetic parameters are usually done outside of the cellular context, and can not be easily scaled-up to a genomic level. It would therefore be valuable to develop methods to assign effective kinetic parameters to transcriptional networks based on in-vivo measurements.

### SUMMARY OF THE INVENTION

The background art does not teach or suggest the analysis of the results of large-scale monitoring of gene expression to examine the relationship between temporal behavior of genes and biological function. The background art also does not teach or suggest mapping biological systems or subsystems on the basis of kinetic expression data in living cells. The background art also does not teach or suggest ordering of genes in expression pathways according to such an analysis of the kinetics of gene expression.

The present invention overcomes these deficiencies of the background art by providing a method for analyzing the temporal behavior of gene expression for a group of genes which are part of a biological system. Preferably, such an analysis enables the order of expression of such genes to be determined. More preferably, the temporal behavior of gene expression is assessed according to the analysis of the kinetics of gene transcription.

According to a preferred embodiment of the present invention, the kinetics of gene transcription are measured according to promoter activity of a plurality of genes. More preferably, such kinetics are measured in a living organism or a portion of such an organism, such as a cell for example. For single-celled organisms, such as bacteria for example, the kinetics may easily be measured for the entirety of the living organism.

Hereinafter, the term “biological system” refers to a group of biologically active molecules which interact for a particular biological structure and/or function, or a plurality of such structures and/or functions. Examples of such biologically active molecules include, but are not limited to, proteins and RNA molecules, or any such group of molecules having a biological function.

According to an embodiment of the present invention, there is provided a method for analyzing the temporal behavior of a plurality of genes for a biological system, comprising: measuring gene expression for the plurality of genes over a period of time, wherein at least a portion of the plurality of genes are wild type genes; and determining an order of expression of the plurality of genes.

Preferably, the measuring is performed for gene expression in a living cell.

Also preferably, the measuring comprises measuring a level of gene transcription according to promoter activity for the plurality of genes.

Preferably, the determining the order comprises: determining an expression profile for the plurality of genes; and comparing the expression profiles. More preferably, the comparing the expression profiles further comprises: clustering a plurality of genes according to similarity in the expression profiles.

Preferably, the measuring the gene expression is performed according to a metric for determining a distance between the genes, the metric being determined according to a correlation of kinetics of the gene expression of each pair of genes. More preferably, the kinetics are measured according to a direct measurement of gene expression. Most preferably, the kinetics are measured according to an indirect measurement of a biological activity associated with the gene expression.

Preferably, the determining the order of expression further comprises: grouping the plurality of genes according to relative distances to form a plurality of groups; ordering the groups of genes according to the relative distances; and ordering genes within each group according to a temporal order of expression of the genes.

More preferably, the grouping of the plurality of genes according to relative distances is performed according to a threshold of relatedness. Most preferably, the grouping of the plurality of genes further comprises: recalculating distances between each pair of groups of genes according to an average distance between genes in each group; and ordering the groups according to the distances.

Also most preferably, the threshold is lowered and at least one group of genes is split into a plurality of smaller groups according to the lowered threshold of distance. Preferably, the groups of genes are ordered according to a dendogram.

Alternatively, the ordering the genes within each group is performed by: determining a relative time of expression of each gene; and ordering the genes according to the relative times of expression.

Preferably, the determining the order of expression further comprises: determining a quantitative value for a parameter for kinetics of the expression for at least one gene. More preferably, the quantitative value is determined for parameters for the plurality of genes. Most preferably, the quantitative values are analyzed to detect at least one regulatory relationship between the plurality of genes. Also most preferably, the determining the order of expression further comprises: constructing a mathematical model according to the at least one regulatory relationship and the quantitative values for the plurality of genes.

The method more preferably further comprises determining an optimized mathematical model for the plurality of genes. Optionally and preferably, the method further comprises optimizing at least one biological process according to the mathematical model. Optionally, the at least one biological process is related to agriculture. Also optionally, optimizing the at least one biological process is applied for treatment of an animal. Preferably, optimizing the at least one biological process is applied for treatment of a non-human animal.

Optionally and preferably, measuring gene expression further comprises: constructing a gene construct comprising a promoter for the gene and a marker gene; and measuring activity of the marker gene according to expression in a cell. Optionally, the marker gene is GFP (green fluorescent protein).

Optionally, the biological system comprises a tissue suspected of cancer, and the method further comprises: determining a gene expression profile for the tissue; and detecting the cancer according to the gene expression profile. More preferably, detecting further comprises staging the cancer.

Optionally and preferably, the biological system comprises a parasite, the method further comprising: determining a gene expression profile for the parasite. More preferably, the method further comprises determining a life cycle stage for the parasite according to the gene expression profile. Also more preferably, the method further comprises determining a treatment for the parasite according to the gene expression profile.

According to another embodiment of the present invention, there is provided a method for analyzing a biological system, comprising: measuring gene expression for a plurality of genes over a period of time to determine kinetics of the gene expression according to a measurement method having a high temporal resolution; selecting at least a subset of genes from the plurality of genes according to the kinetics of the gene expression; and determining a temporal relationship between genes in the subset of genes for analyzing the biological subsystem, wherein the temporal relationship comprises at least a partial order of gene expression for the plurality of genes.

According to yet another embodiment of the present invention, there is provided a method for analyzing the temporal behavior of a biological system, the biological system being associated with a plurality of genes, the method comprising: measuring gene expression for the plurality of genes over a period of time according to a measurement method having a high temporal resolution, wherein a biological function of at least a portion of the plurality of genes is substantially unaltered by the measurement method during the period of time; determining a relative distance between at least the portion of the plurality of genes according to the measurement method; and ordering at least the portion of the plurality of genes according to the relative distance.

According to still another embodiment of the present invention, there is provided a method for analyzing the temporal behavior of a plurality of genes for a biological system, comprising: providing a metric for determining a distance between each pair of genes; measuring gene expression for the plurality of genes over a period of time, wherein the measurement method is capable of measuring the gene expression without substantially altering a biological function of the plurality of genes during the period of time; grouping the plurality of genes according to relative distances to form a plurality of groups; ordering the groups of genes according to the relative distances; and ordering genes within each group according to a temporal order of expression of the genes.

Preferably, the metric is determined according to a correlation of kinetics of the gene expression of each pair of genes. More preferably, the kinetics are measured according to a direct measurement of gene expression. Most preferably, the kinetics are measured according to an indirect measurement of a biological activity associated with the gene expression.

Alternatively, the grouping the plurality of genes according to relative distances is performed according to a threshold of relatedness. Preferably, the grouping of the plurality of genes further comprises: recalculating distances between each pair of groups of genes according to an average distance between genes in each group; and ordering the groups according to the distances. More preferably, the threshold is lowered and at least one group of genes is split into a plurality of smaller groups according to the lowered threshold of distance. Most preferably, the groups of genes are ordered according to a dendogram.

Alternatively, the ordering the genes within each group is performed by: determining a relative time of expression of each gene; and ordering the genes according to the relative times of expression.

According to still another embodiment of the present invention, there is provided a method for constructing a mathematical model of expression of a plurality of genes in a biological system, the method comprising: measuring gene expression for the plurality of genes over a period of time to determine kinetics of the gene expression according to a measurement method having a high temporal resolution; determining a quantitative value for a parameter for kinetics of the expression for the plurality of genes; analyzing the quantitative values to detect at least one regulatory relationship between the plurality of genes; and constructing the mathematical model according to the at least one regulatory relationship and the quantitative values for the plurality of genes.

Preferably, the determining the quantitative value further comprises: determining an expression profile for at least one gene of the plurality of genes; and extrapolating from the expression profile and the measured gene expression to calculate the quantitative values for the plurality of genes.

More preferably, the expression profile further comprises a profile of concentrations of a protein coded by the at least one gene.

According to yet another embodiment of the present invention, there is provided a method for determining kinetic parameters of a gene regulation system for a plurality of genes, the method comprising: measuring gene expression for the plurality of genes over a period of time, wherein the measurement method is capable of measuring the gene expression without substantially altering a biological function of the plurality of genes during the period of time; and analyzing the gene expression according to the measurement method to determine the kinetic parameters. Preferably, the method further comprises constructing the mathematical model according to the kinetic parameters.

According to another embodiment of the present invention, there is provided a method for determining time-dependent regulator activity based on transcriptional activity of a plurality of regulated genes, the genes being regulated through a regulator, the method comprising: measuring the transcriptional activity for the plurality of genes over a period of time according to a measurement method, the measurement method being capable of measuring the transcriptional activity without substantially altering a biological function of the plurality of genes during the period of time; and determining time-dependent activity of regulation through the regulator.

Preferably, the method further comprises: measuring activity of a regulatory protein through the time-dependent activity of regulation through the regulator, wherein the regulatory protein binds to the regulator.

According to another embodiment of the present invention, there is provided a method for detecting an influence of a pattern of a plurality of factors on a plurality of stocks in a stockmarket, the method comprising: measuring prices for the plurality of stocks over a period of time at a plurality of time points; determining at least a presence of each of the plurality of factors at each time point; and determining the pattern according to the at least a presence of the plurality of factors and the prices to detect a potential correlation.

### BRIEF DESCRIPTION OF THE DRAWINGS

The invention is herein described, by way of example only, with reference to the accompanying drawings, wherein:

FIG. 1 shows the genetically defined hierarchy of flagellar operons in Escherichia coli;

FIG. 2 shows a flowchart of an exemplary method according to the present invention for analyzing expression kinetics;

FIG. 3A shows the fluorescence of flagella reporter strains as a function of time, normalized by the maximal fluorescence of each strain, while FIG. 3B shows the fluorescence of flagella reporter strains as a function of time for two experimental conditions;

FIG. 4 shows the kinetic classification of the flagellar operons according to the results of the method of the present invention;

FIG. 5 shows the bacterial SOS DNA repair system. DNA damage is sensed by RecA, which induces autocleavage of the repressor LexA. LexA binds to the promoters of the SOS operons, including its own promoter and that of RecA;

FIG. 6: (a) Fluorescence of SOS reporter strains as a function of time following UV irradiation. (b) SOS Promoter activity, rate of green fluorescent protein production per OD unit. E. coli strain AB 1157 with SOS reporter plasmids was grown in 96-well plates at 37° C. in a multiwell fluorimeter, a UV dose of 5Jm⁻² was given at mid exponential growth (t=0). (c) Unsmoothed GFP fluorescence (background subtracted) for repeat experiments performed on different days. Each point represents one time point, for a total of 99 time points per operon for 8 operons. A perfect repeat would be on the x=y diagonal, also shown are parallel diagonal lines representing 10% errors. The mean error is 10.4%. UV=5Jm⁻²;

FIG. 7 shows promoter activity (solid line) and promoter activity predicted from the kinetics of a single promoter (uvrA) using the βᵢ and kᵢ values and eq. 3 (dashed line) at UV=5Jm⁻². The promoter activity of recA and lexA is multiplied by 0.25;

FIG. 8 shows the effective relative repressor concentration A(t) at UV=5Jm⁻² (solid line) and at UV=20Jm⁻² (dotted line). The cell cycle time is 45 min. Relative LexA protein levels measured using immunoblots, at UV=5Jm⁻² (asterisk) and at UV=20Jm⁻² (circle) in the same strain and conditions;

FIG. 9 shows the time point kinetics of 96 promoters over 12 h (twelve hours) of growth, with the y-axis showing GFP (green fluorescent protein) activity as expressed in O.D. units, and the x-axis showing time;

FIG. 10 shows accuracy of repeat experiments, in which average errors between repeat experiments were less than 10%; GFP activity is shown in the graph on the left, while O.D. is shown for the graph on the right;

FIG. 11 shows a schematic diagram of the pathway of the arginine biosynthesis genes;

FIG. 12 shows the effect of growing cells in media which either contained or did not contain arginine;

FIG. 13 shows that addition of cysteine leads to down regulation of Cys genes;

FIGS. 14A and 14B show that the order of gene expression of the arginine pathway for high temporal resolution analyses matches the order of the pathway, in which FIG. 14A shows argF, argI, argG and argH; while FIG. 14B shows argA-E and argR;

FIG. 15 shows that the order of gene expression of the serine biosynthetic pathway for high temporal resolution analyses matches the order of the pathway;

FIG. 16 shows that the order of gene expression of the methionine biosynthetic pathway for high temporal resolution analyses matches the order of the pathway;

FIG. 17 shows expression of the genes argA, argCBH, argD and argE; x-axis shows level of expression as LUX/OD, y-axis shows time;

FIG. 18 shows that enzymes involved in early stages of linear pathways have faster rise times visible when checking normalized gene expression for the arginine biosynthetic pathways (the data shown in FIG. 17 were normalized for this graph);

FIG. 19 shows the relationship between rise time and maximal response for a transcriptional system for arginine;

FIG. 20 shows expression of the genes serA, serB, and serC (left), and metA, metB and metC (right); x-axis shows level of expression as GFP/OD, y-axis shows time;

FIG. 21 shows the relationship between rise time and maximal response for a transcriptional system for serine (left) and methionine (right); and

FIG. 22 shows a mathematical description of the arginine biosynthetic pathway.

### DESCRIPTION OF THE PREFERRED EMBODIMENTS

The present invention provides a method for analyzing the temporal behavior of gene expression for a group of genes which are part of a biological system. Preferably, such an analysis enables the order of expression of such genes to be determined. More preferably, the temporal behavior of gene expression is assessed according to the analysis of the kinetics of gene transcription.

According to a first embodiment of the present invention, the method features, in a first stage, the creation of a plurality of clusters according to the temporal behavior of gene transcription in the system; ordering of the clusters; and then ordering of the gene transcription for those clusters which feature a plurality of genes.

According to a preferred embodiment of the present invention, the kinetics of gene transcription are measured according to promoter activity of a plurality of genes. More preferably, such kinetics are measured in a living organism or a portion of such an organism, such as a cell for example. For single-celled organisms, such as bacteria for example, the kinetics may easily be measured for the entirety of the living organism.

The operation of the method of the present invention is supported by three exemplary biological systems, the temporal behavior of which was analyzed according to the present invention, for the purposes of illustration only and without any intention of being limiting. The first biological system featured the flagellum of E. coli bacteria, such that the present invention was used to analyze the order of transcription of the genes which code for the proteins for constructing this biological structure. The second biological system featured the SOS bacterial system of E. coli bacteria, as described in greater detail below. The third biological system featured the arginine synthesis pathway of E. coli bacteria, also as described in greater detail below.

With regard to the flagellum, it was chosen as a model biological subsystem for assessing the efficacy of the present invention, as they represent a particular biological structure with a clearly defined biological function. Furthermore, the proteins of which flagella are composed have also been extensively studied, thereby enabling the results of the method of the present invention to be more clearly interpreted. The experimental methods and results are described with regard to Example 1 below. The present invention was able to determine the correct order of gene expression from transcriptional data, according to one exemplary embodiment of the method of the present invention. As previously described, this embodiment features the creation of a plurality of clusters according to the temporal behavior of gene transcription in the system; ordering of the clusters; and then ordering of the gene transcription for those clusters which feature a plurality of genes. Without wishing to be limited in any way, Example 1 centers on the use of dendograms for ordering the clusters as an illustrative method.

The use of the method of the present invention results in a strikingly detailed temporal program of expression that correlates with the functional role of the SOS genes and is driven by a hierarchy of effective kinetic parameter strengths for the various promoters. The calculated parameters can be used to determine the kinetics of all SOS genes given the expression profile of just one representative, allowing a significant reduction in complexity. The concentration profile of the master SOS transcriptional repressor can be calculated, demonstrating that relative protein levels may be determined from purely transcriptional data. This opens the possibility of assigning kinetic parameters to transcriptional networks on a genomic scale.

According to preferred embodiments of the present invention, the method provides an accurate measurement of the temporal behavior of gene transcription as it is performed, such that the creation of mutants or the addition of toxic chemicals (or other toxic treatments, such as radiation for example) to the system are not required, although they may optionally be used. Such methods of epistatic analysis are preferably not required, as they may perturb the biological system in such a manner as to distort the true functioning of this system.

By contrast, currently available methods for the analysis of expression kinetics are not preferred as they require such epistatic analysis, which may result in the analysis of a “broken system”, rather than providing an accurate picture of the biological functions of the system. Furthermore, such invasive analysis is difficult to perform on a large scale, as the functioning of the biological system must be substantially or completely stopped in order for the analysis to be performed. Therefore, the present invention has a clear advantage over currently available analysis methods, as only the present invention may optionally be used to analyze the behavior of a wild type biological system.

Accuracy of the system is also described below with regard to Example 4, which demonstrates the high degree of accuracy of the present invention, and also the greater accuracy of the present invention than existing background art methods for measuring gene expression.

### Example 1

### Analysis Method

The following algorithm was used for the implementation of an exemplary method according to the present invention, for analyzing the temporal behavior of gene expression for a group of genes which are part of a biological system. Preferably, such an analysis enables the order of expression of such genes to be determined. More preferably, the temporal behavior of gene expression is assessed according to the analysis of the kinetics of gene transcription. The exemplary method according to the present invention uses a clustering algorithm to determine the order of expression for the purposes of illustration only, as any other suitable algorithm for the analysis of the temporal behavior of gene expression could optionally be used, as could easily be selected by one of ordinary skill in the art.

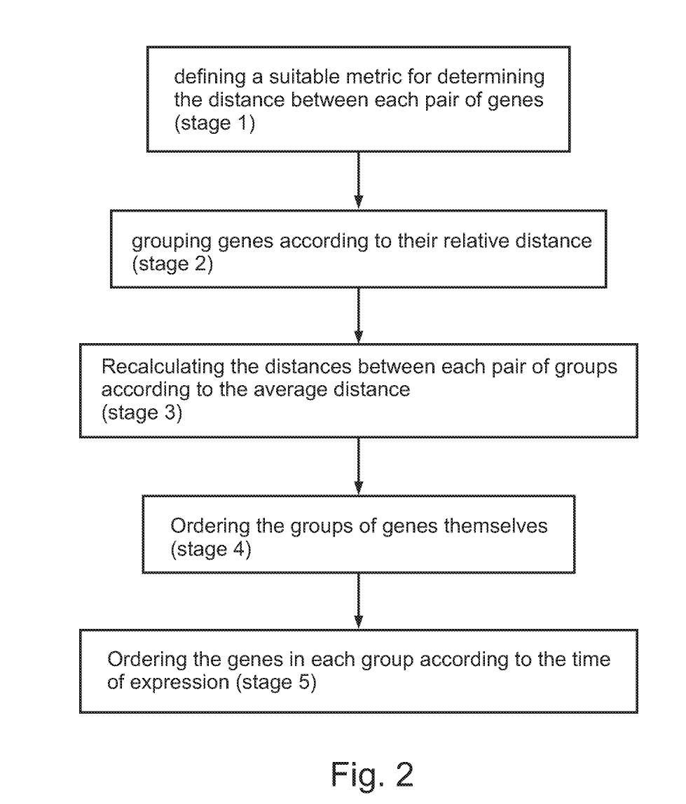

As shown with regard to FIG. 2, the exemplary method according to the present invention starts with the definition of a suitable metric for determining the distance between each pair of genes. By “distance”, it is meant the relationship between these genes with regard to their temporal behavior, in terms of the kinetics of gene expression over time. As a preferred but optional, non-limiting example, the distance metric is preferably determined as follows:

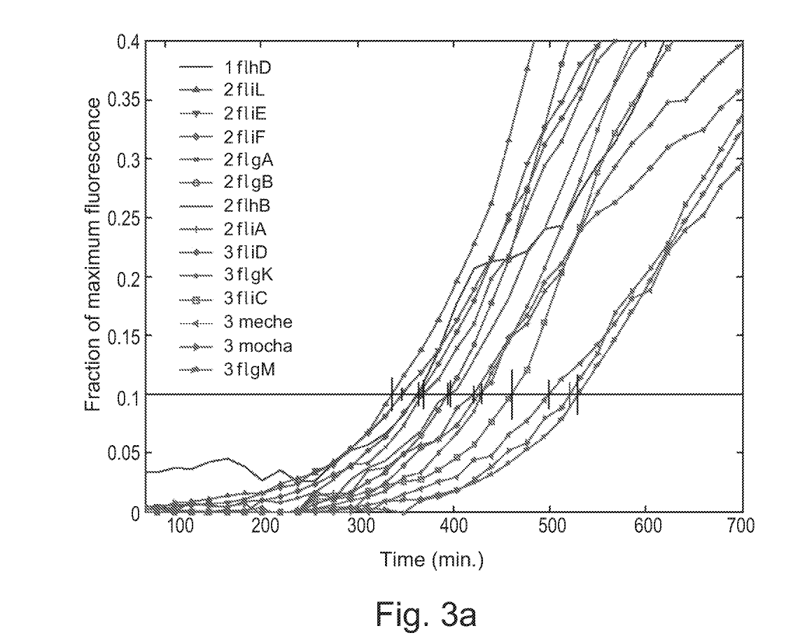

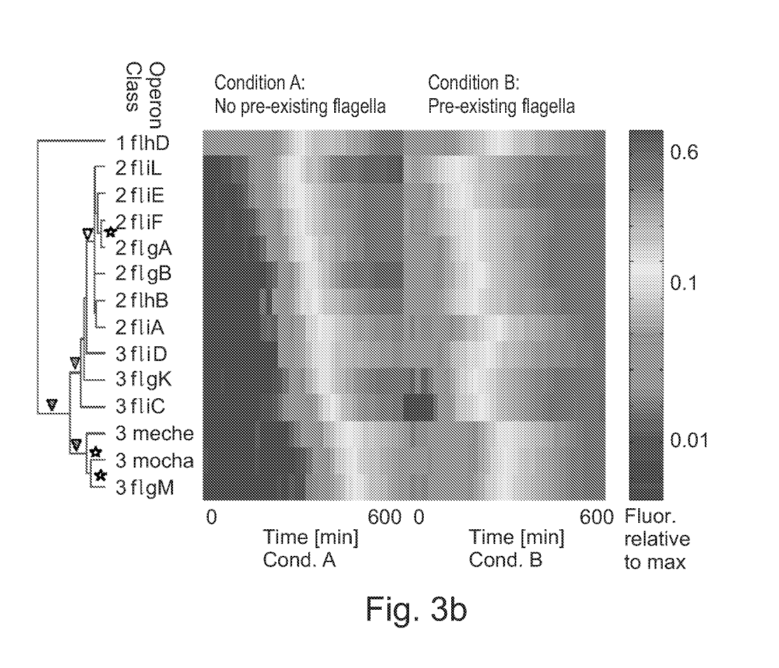

d(i,j)=l−corr(i,j)

in which the distance between two genes i, j (d (i, j)) is equal to one minus the correlation of the temporal behavior of these genes (corr (i, j)). This correlation is preferably determined by comparing the kinetics of gene transcription, for example according to the behavior of a reporter gene linked to a promoter for the gene of interest, as described in greater detail below with regard to FIG. 3. If the correlation between the two genes i, j is high, or close to one, then the distance between the two genes is low, or close to zero.

In stage 2 of the method of FIG. 2, the genes are preferably initially grouped according to their relative distances. More preferably, the groups are determined according to a threshold of relatedness, such that the genes more preferably have a distance that is lower than a given threshold in order to be placed in one group.

In stage 3, the distances between each pair of groups are then preferably recalculated according to the average distance between the members of the groups in the pair. Stages 2 and 3 may optionally and preferably be repeated, and more preferably are repeated with the threshold distance for relatedness being lowered after each repetition in order to optionally split larger groups into smaller groups. Also, optionally and most preferably, the resultant ordering may be expressed in the form of a dendogram or hierarchical tree. For such an ordering, the smallest groups would be the leaves of the tree (at its ends), while the larger groups would form branches higher up in the tree or hierarchy.

In stage 4, the groups of genes themselves are preferably ordered, according to the average time for expression of the genes in each group, in order to determine which group is expressed first, which group is expressed second and so forth. Such ordering is preferably performed with the larger groups (those groups that are higher up in the hierarchy or tree) first, before the smaller groups. The average time for expression is preferably determined by measuring the expression kinetics, and then using some type of function, such as

∫log ƒ(t)

for example. The order of the groups is then preferably determined according to the time of expression. For example, if the groups are being ordered in a tree, then those groups with earlier times of expression are preferably placed to the left of other groups, until ordering is complete between the groups.

In stage 5, the genes in each group are preferably ordered according to the time of expression, in order to determine the overall order of gene expression in the biological system. A similar function may optionally be used for determining the order of expression of the genes within a group.

Once the genes within each group have been ordered, the overall order of gene expression has been determined, according to the combination of ordering within each group and ordering of the groups.

According to preferred embodiments of the present invention, the kinetics of gene transcription are optionally and preferably determined according to the behavior of a reporter gene linked to a promoter for the gene of interest, as described in greater detail below with regard to FIG. 3. Alternatively or additionally, any other method for measuring gene transcription may optionally be used. Preferably, such a method has a high temporal resolution. The degree of resolution is therefore sufficient to distinguish between the steps of gene transcription in the functioning of the biological system, and may optionally be a few minutes for bacteria, for example.

Also preferably, the method provides an accurate measurement of the temporal behavior of gene transcription as it is performed, such that the creation of mutants or the addition of toxic chemicals (or other toxic treatments, such as radiation for example) to the system are not required, although they may optionally be used. Such methods of epistatic analysis are preferably not required, as they may perturb the biological system in such a manner as to distort the true functioning of this system.

According to other preferred embodiments of the present invention, in addition to measuring the expression kinetics with a high degree of temporal resolution, preferably the temporal behavior of a gene in the biological system is perturbed slightly in order to more carefully determine the role of such a gene in that system. Such a perturbation may include an increase or a decrease in the expression, and/or earlier or later expression of the gene. Such a perturbation may optionally be used in order to determine whether the gene belongs to that biological system, for example. However, the present invention may also optionally be used for the analysis of the behavior of a plurality of gene simultaneously.

According to other optional but preferred embodiments of the present invention, accurate high temporal-resolution measurement of gene activities, such as promoter activities for example, are used to assign effective kinetic parameters within a mathematical model of the network of genes in the biological system. This is demonstrated below by using a well-defined network, the SOS DNA repair system of Escherichia coli. A detailed temporal program of expression was determined according to the method of the present invention, that correlates with the functional role of the SOS genes and that is driven by a hierarchy of effective kinetic parameter strengths for the various promoters. The calculated parameters can be used to determine the kinetics of all SOS genes given the expression profile of just one representative, allowing a significant reduction in complexity. The concentration profile of the master SOS transcriptional repressor can be calculated, demonstrating that relative protein levels may be determined from purely transcriptional data. This opens the possibility of assigning kinetic parameters to transcriptional networks on a genomic scale.

### Example 2

### Flagella as a Biological Subsystem

Flagella were chosen as a model biological subsystem for assessing the efficacy of the present invention, as they represent a particular biological structure with a clearly defined biological function. Furthermore, the genes and proteins of which flagella are composed have also been extensively studied, thereby enabling the results of the method of the present invention to be more clearly interpreted. It should be noted that flagella are intended as an illustrative, non-limiting example of such a biological subsystem, as the method of the present invention is clearly useful for the analysis of the behavior of a wide variety of such biological subsystems and systems.

Although optionally any marker for gene transcription may have been used in order to measure the kinetics of gene expression, promoter activity was chosen as a non-limiting illustrative example of such a marker for the demonstration of the method of the present invention, because reporter plasmids may be used for the determination of promoter activity. However, this is intended as a non-limiting example only, as any other marker having a sufficiently high temporal resolution and accuracy. For example, promoter activity that is linked to a reporter gene typically has a high signal to noise ratio. Other examples include but are not limited to, protein level reporter markers such as protein fusions, RNA level reporters such as hybridization based assays, as well as any other such marker or reporter for cellular activity that has temporal resolution and accuracy.

For the present example, the genes in the pathway were ordered according to the method of the present invention without dependence on mutant strains. However, optionally the present invention also encompasses the use of such mutant strains, which confers added advantages over the background art. The observed temporal program of transcription was much more detailed than was known in the background art, and was associated with multiple steps of flagella assembly.

### Experimental Methods

Real-time monitoring of the transcriptional activation of the flagellar operons was performed with a panel of 14 reporter plasmids in which green fluorescent protein (GFP) (6) is under the control of one of the flagellar promoters (7). Specifically, two bacterial strains were used, RP437 (19) and YK410 (20), which are E. coli K12 strains that are wild type for motility and chemotaxis. The polymerase chain reaction was used to amplify the flagellar promoter regions using primers designed from the MG1655 genome sequence (21). The promoter region coordinates used were flhD (1976454-1976212), flgB (1130044-1130245), flgA (1130245-1130044), fliA (2000123-1999779), fliD (2001594-2001916), fliC (2001916-2001594), fliE (2011261-2010998), fliF (2010998-2011261), fliL (2017491-2017644), meche (1970893-1970676), mocha (1975301-1975161), flgM (1129471-1129331), flgK (1137467-1137656), and flhB (1964392-1964190).

Reporter plasmids were constructed by subcloning these promoter regions into a Bam HI site upstream of a promoterless GFP on the low-copy vector pCS21. pCS21 was constructed by replacing the luciferase gene of pZS21-luc (22) with a DNA fragment containing the GFPmut3 (6) gene. Promoter identity was verified by sequencing. There was no observable effect of the plasmids on swimming motility as assayed on soft agar plates [performed as described (23)], suggesting that the system can compensate for the extra promoter copies introduced by these low-copy plasmids (data not shown). There were no measurable differences in the growth rate of the reporter strains, with the exception of the reporters for meche, mocha, and flgM, which show a somewhat faster growth in culture (data not shown).

Continuous time courses from living cells grown in a multiwell plate fluorimeter were measured as follows (14). Cultures (2 ml) inoculated from single colonies were grown 16 hours in Tryptone broth (Bio 101, Inc.) with kanamycin (25 μg/ml) at 37° C. with shaking at 300 rpm. The cultures were diluted 1:600 or 1:60 into defined medium [M9 minimal salts (Bio 101, Inc.)+0.1 mM CaCl₂+2 mM MgSO₄+0.4% glycerol+0.1% casamino acids+kanamycin], at a final volume of 150 μl per well in flat-bottomed 96-well plates (Sarsteadt 82.1581.001). The cultures were covered by a 100-μl layer of mineral oil (Sigma M-3516) to prevent evaporation during measurement. Cultures were grown in a Wallac Victor2 multiwell fluorimeter set at 30° C. and assayed with an automatically repeating protocol of shaking (1 mm orbital, normal speed, 180 s), fluorescence readings (filters F485, F535, 0.5 s, CW lamp energy 10,000), and absorbance (OD) measurements (600 nm, P600 filter, 0.1 s). Time between repeated measurements was 6 min. Background fluorescence of cells bearing a promoterless GFP vector was subtracted. RP437 was the parental strain of all reporter strains, except flhDC, for which the signal was below background at early time points, and thus YK410 was used. Similar timing and temporal ordering of the flagellar operons was observed in this strain. The high temporal resolution of the present system benefits from the apparent rapid activation of GFP in bacteria (24, 25) as compared with reported times for folding and oxidation of the chromophore in vitro, 10 min and 1 hour, respectively (26).

### Results

The previously described experimental method enabled previous timing studies that depended on lacZ fusions to be extended to up to four operons (8, 9). Use of GFP eliminates the need for cell lysis required for lacZ and DNA microarray studies (10-13). Therefore, the present experimental method enables continuous time courses from living cells grown in a multiwell plate fluorimeter to be measured. Average errors between repeat experiments were less than 10%, compared with errors of at least twofold often associated with expression assays requiring cell lysis and manipulation (10-12).

The flagella system is turned on during the exponential phase of growth. Clustering the fluorescence levels of the operons (FIG. 3A) according to similarity in their expression profiles (10-13) showed that they fall into clusters that correspond to the genetically defined classes 1 and 2 (FIG. 3B). Three of the six class 3 operons are close to the compact class 2 cluster, and the other three are in a separate cluster. This separation is based mainly on different coordinated responses of the operon classes.

FIG. 3A shows the fluorescence of flagella reporter strains as a function of time, normalized by the maximal fluorescence of each strain. An average of five experiments in growth condition A are shown (bars, SD). Class 1, 2, and 3 operons are marked in blue, red, and green, respectively.

FIG. 3B shows the fluorescence of flagella reporter strains as a function of time for two experimental conditions. Log intensity of each promoter, normalized by its maximal value in each experiment, scales from blue (low) to red (high). Operons are arranged according to the temporal clustering results. The first 630 minutes of each experiment, for two growth conditions, with and without preexisting flagella, are shown.

Growth condition A was performed as follows. Stationary-phase cultures with two to five flagella per cell (29) were diluted 1:600 into fresh medium; induction of new flagella begins after about three to four generations, and thus old flagella were diluted out by cell division to a degree that most cells have no preexisting flagella. Growth condition B was performed as follows. Overnight cultures were diluted 1:60. The flagellar operons were turned on within one cell generation so that old flagella were present. The presence or absence of preexisting flagella was verified by microscopic observation of cell motility as described (23).

The dendogram shows hierarchical gene clustering and temporal order. The statistical significance (P value) for temporal ordering at each splitting was determined by the fraction of times that a larger |t₁−t₂| value was found upon clustering and labeling 1000 randomized data sets generated by randomly permuting the gene coordinates at each time point. Similarly, a P value for clusters was determined by the fraction of times that a larger splitting distance occurred in the randomized data sets. Clusters with significance P<0.001 are marked with filled triangles; P≈0.01 with an open triangle; and P>0.01, no triangles. Temporal ordering of all tree splittings is significant (P<0.01), except the splittings marked with a star.

To determine the timing order, the method of the present invention was extended with an optional but preferred temporal labeling procedure that hierarchically orders the clusters according to the relative timing of their average expression profiles. Log fluorescence of each reporter strain, normalized by its maximum for each experiment, was set to zero mean and variance one, and clustered by means of a standard single-linkage algorithm with a Euclidean metric (Matlab 5.3, Mathworks) (15). In general, clustering algorithms do not specify an ordering of the clusters.

In the resulting dendograms, as the data are split hierarchically into a tree, pairs of subtrees in each splitting are placed in an arbitrary order. To define the temporal order of expression, each splitting was first considered from the top down and computed the average log fluorescence (normalized by the maximal fluorescence) for the two subtrees, log(ƒ₁) and log(ƒ₂). Next, tᵢ=−∫log [ƒᵢ(t)]dt was computed (generally the earlier a sigmoidal curve rises, the smaller its tᵢ. Since log fluorescence is used, the initial rise timing is emphasized.) The subtree with the smaller tᵢ was then positioned to the left. The present algorithm was able to correctly order simulated gene cascades.

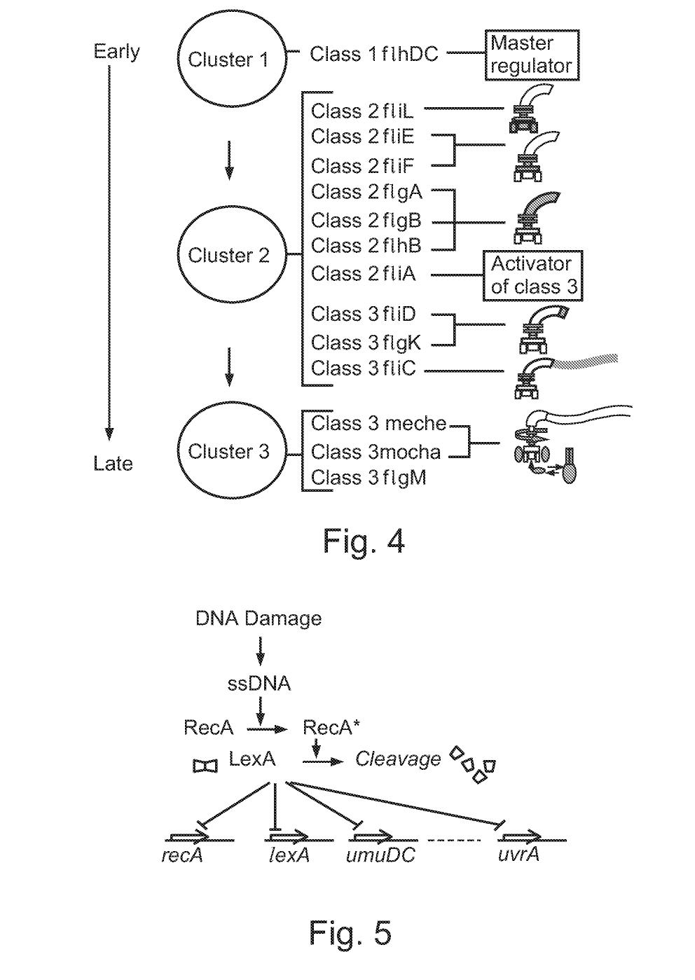

FIG. 4 shows the kinetic classification of the flagellar operons according to the results of the method of the present invention. The three clusters and the operons within each cluster are arranged by their relative timing according to the temporal clustering results. Positions of the corresponding gene products in the flagellum (1) are indicated in green.

The method of the present invention arranged the operons in the order: class 1 followed by class 2 followed by class 3 (8, 9) (FIG. 3B). Within the class 2 cluster, the promoters were turned on sequentially, with significant delays, in the order fliL, fliF, fliF, flgA, flgB, flhB, and fliA (FIG. 3). The observed order corresponds to the spatial position of the gene products during flagellar motor assembly, going from the cytoplasmic to the extracellular sides (1, 2) (FIG. 4). The fliL operon genes form the cytoplasmic C ring, and fliE and fliF genes form the MS ring in the inner membrane, thought to be the first assembled structure (1). The flgA, flhB, and flgB genes participate in the export and formation of the periplasmic rod, the distal rings in the outer membrane, and the extracellular hook. The transcription factor responsible for turning on class 3 genes, fliA, is the last class 2 gene to turn on.

A separation of class 3 genes into two kinetic groups was seen, with the filament structural operons flgK, fliD, and fliC activated first, and flgM and the chemotaxis operons meche and mocha going on only after a substantial delay (FIG. 4). Thus, the hardware for the flagellar propeller is expressed before the chemotaxis navigation system (FIG. 4). The genes for motor torque generation, motAB in the mocha operon, are in the late class 3 group, and indeed, it has been shown that they can be functionally incorporated long after motors are assembled (16, 17).

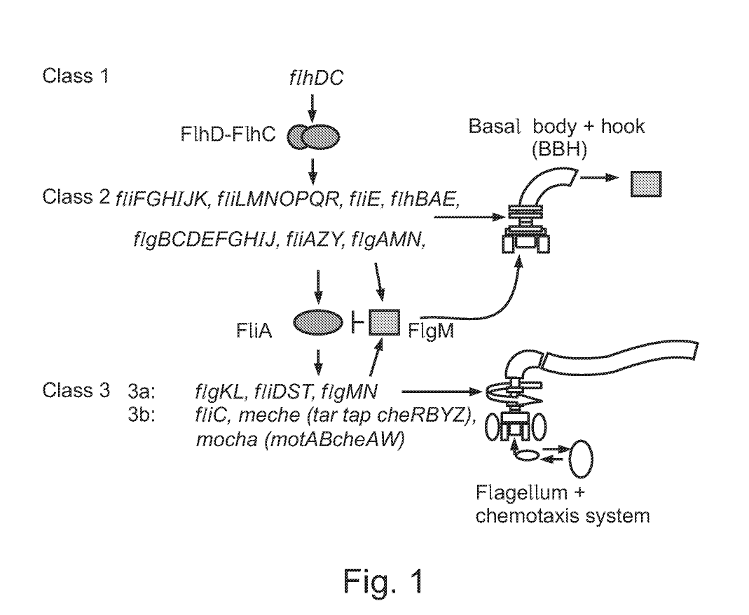

When flagella were induced in cells with no preexisting flagella, a temporal separation between most class 2 genes and class 3 genes was observed (FIG. 3B, condition A); whereas in cells with preexisting flagella, the delay between class 2 and the early class 3 genes decreased drastically (FIG. 3B, condition B). This probably reflects the checkpoint in flagella biosynthesis (FIG. 1). When preexisting flagella are present, newly synthesized FlgM is exported from the cells even before new basal bodies are completed. This frees FliA to turn on class 3 genes at an earlier time. Such memory effects may be a general kinetic signature of regulatory checkpoints.

Without wishing to be limited to a single hypothesis, it is possible that the mechanism underlying the temporal order of promoter activation within classes 2 and 3 involves ranking the DNA regulatory sites in the promoter regions of the operons in affinity. As the concentration of the relevant transcription factor (FlhDC, FliA) gradually increases in the cell, it first binds and activates the operons with the highest affinity sites, and only later does it bind and activate operons with lower affinity sites.

The standard, background art genetic method of pathway analysis, which requires the use of mutant cells, suffers from the limitation that conclusions drawn from mutant cells sometimes apply to a physiological state far from wild-type. The method of the present invention, in contrast, enables cells with an intact regulatory system to be probed, rather than mutant cells. For example, class 3 operons were subdivided by mutant analysis into class “3a” and class “3b” (FIG. 1), based on residual expression in a fliA mutant of class 3a but not 3b operons (1). This mutant may exemplify a situation never reached by wild-type cells (high FlhDC but no FliA). The present kinetic subdivision of class 3 operons into early and late temporal groups provides a functionally reasonable order.

Again without wishing to be limited by a single hypothesis, the precise order of transcription of the various operons may not be essential for assembling functional flagella. This is suggested by complementation experiments in which the motility of flagella mutants was rescued by expression of the wild-type gene from a foreign promoter (1). The detailed transcription order could, however, function to make flagella synthesis more efficient, because parts are not transcribed earlier than needed. From the viewpoint of reverse engineering, this may be exploited to decipher detailed assembly steps from transcription data.

### Example 3

### SOS System

This Example again analyzes genetic expression data in order to determine the effective kinetic parameters for a transcriptional network of a plurality of genes. The experimental data described below is based on accurate high temporal-resolution measurement of promoter activities from living cells using green fluorescent protein reporter plasmids. The transcriptional network which is used for the present experiments is a well-defined network, the SOS DNA repair system of Escherichia coli. The promoter is a non-limiting example of a regulator, while proteins that bind to the regulator are non-limiting examples of regulatory proteins.

The SOS DNA repair system includes about 30 operons regulated at the transcriptional level. A master repressor (LexA) binds sites in the promoter regions of these operons. One of the SOS genes, RecA, acts as a sensor of DNA damage: by binding to single-stranded DNA it becomes activated and mediates LexA autocleavage. The drop in LexA levels causes the de-repression of the SOS genes (FIG. 5). Once damage has been repaired or bypassed, the level of activated RecA drops, LexA accumulates, represses the SOS operons and the cells return to their original state.

This Example demonstrates that effective kinetic parameters can be used to detect SOS genes with additional regulation, to capture the temporal transcriptional program and to calculate the concentration profile of the regulatory protein.

Methods

Plasmids and Strains: Promoter regions were amplified from MG1655 genomic DNA using PCR and the following start and end coordinates for the primers taken from the sequenced E. coli genome (30): uvrA (4271368-4271753), uvrD (3995429-3995664), lexA (425-4491-425-4751), recA (2821707-2821893), ruvA (1943919-1944201), polB (65704-65932), umuD (1229552-1230069), uvrY (1993282-1993900), lacZ (365438-365669). This includes the entire region between ORFs with an additional 50-150 base pair into each of the flanking ORFs. The promoter regions were cloned using XhoI and BamHI into the reporter plasmids, upstream of a promoterless GFPmut3 gene in a low copy pSC101 origin plasmid as described (31). The plasmids were transformed into the E. coli strain AB1157 [argE3, his4, leuB6, proA2, thr1, ara14, galK2, lacY1, mtl1, xy15, thi1, tsx33, rpsL31, supE44].

Culture and measurements: Cultures of strain AB1157 (1 ml) inoculated from glycerol frozen stocks were grown for 16 hr in LB medium with kanamycin (25 μg/ml) at 37° C. with shaking at 250 rpm. The cultures were diluted 1:100 into defined medium (32) (M9 supplemented with thiamine (10 μg/ml), glucose (2 mg/ml), MgSO₄ (1 mM), MgCl₂ (0.1 mM), thymine (20 μg/ml), each of the 20 amino acids except tryptophan (50 μg/ml)+25 μg/ml kanamycin), at a final volume of 100 μl per well in flat-bottom 96 well plate (Sarsteadt). The cultures were covered with an adhesive pad to prevent evaporation and grown in a Wallac Victor2 multiwell fluorimeter at 37° C. (unless otherwise noted), set with an automatically repeating protocol of shaking (2 mm orbital, normal speed, 30 sec, 3 min delay).

When the cultures reached mid exponential growth (OD₆₀₀=0.03) they were irradiated with ultraviolet (UV) light at 254 nm with a low-pressure mercury germicidal lamp at levels of 5 or 20 Jm⁻². After addition of 150 μl mineral oil (SigmaM-3516) per well (to prevent evaporation) the plate was returned to the fluorimeter with a second repeated protocol that included shaking (2 mm orbital, normal speed, 30 sec), absorbance (OD) measurements (600 nm filter, 1 sec) and fluorescence readings (filters 485 nm, 535 nm, 0.5 sec, CW lamp energy 10000). Time between repeated measurements was 3 min. Background fluorescence of cells bearing a promoterless GFP vector was subtracted. Growth rate was similar to the promoterless GFP reporter strain.

The present results were obtained with kinetics measurements for 2 cell cycles following DNA irradiation. In experiments that tracked the promoter activity for longer times, an unexpected second peak of promoter activity was found (not shown), which occurs after about two and a half cell cycles. This peak includes only a subset of the SOS promoters, and is thus probably not explained only by a second minimum in LexA levels. It does not appear in operons unrelated to the SOS system, and is thus unlikely to result from global changes in transcription. The second peak may represent the influence of an additional, uncharacterized transcription factor.

The influence of the UV irradiation on plasmid copy number: Plasmids were extracted using a miniprep kit (Qiagen) from an irradiated culture (2 h after a dose of UV=550Jm⁻²) and from an unirradiated control culture. The plasmids were transformed into RP437 CaCl₂ heat-shock competent bacteria. 100 μl from the transformation reaction was plated on LB+25 μg/μl kanamycin. Both irradiated and control cultures produced the same number of colonies (within 5% error), suggesting that the plasmid copy number is not influenced by UV irradiation.

Parameterization algorithm I—trial function: The present study deals with a simple network architecture, where all operons are under negative control by a single repressor. This is modeled using a simple binding of the repressor to a regulatory DNA site in each operon, resulting in a Michaelis-Menten form Eq. (2). In the case where the regulator is an activator, and not a repressor, the appropriate trial function is:

$${X}_{ij} ( t ) = \frac{{β}_{i} {\hat{A}}_{j} ( t ) / {\hat{k}}_{i}}{1 + {\hat{A}}_{j} ( t ) / {\hat{k}}_{i}} .$$

This case is described by the present use of Eq. (2) by simply using the transformations: Â(t)=1/A(t) and \hat{k}ᵢ=1/kᵢ. An extension of Eq. (2) to the case of cooperative binding would be

$${X}_{ij} ( t ) = \frac{{β}_{i}}{1 + {( {A}_{j} ( t ) / {k}_{i} )}^{Hi}} .$$

This allows a different effective Hill coefficient Hi for each operon. This form captures both cooperativity, and the possibility that a regulator is a repressor for some genes and an activator for others, where Hi>0 corresponds to repression and Hi<0 to activation. In principle, it should be evident from the data whether different operons are regulated with different signs by the same regulator, because they will tend to have anti-correlated profiles. The optimization algorithm described below can be generalized to include a Hill-type cooperativity for each promoter. The present data for the repressor protein levels suggest that there may be no significant cooperativity in the repressor action.

Parameterization Algorithm II—Data Preprocessing:

The raw GFP and OD signals were smoothed using a hybrid Gaussian-median filter with a window size of 5 measurements (33). Promoter activity is given by Eq. (1), Xᵢ(t)=(dGᵢ(t)/dt)/ODᵢ(t). The activity signal was then smoothed by a polynomial fit (6-th order) to log(Xᵢ(t)). This captures the dynamics well, while removing the noise inherent in the differentiation of noisy signals. Finally, the data for all experiments were concatenated and normalized by the maximal activity for each operon.

Parameterization Algorithm III—Parameter Determination:

To determine the parameters in Eq. (2) based on experimental data, the equation was first transformed to a bilinear form using 1/x(i,t)=u(i,t)=a(i)A(t)+b(i), where a(i)=1/β(i)k(i), b(i)=1/β(i). In this bilinear form, the matrix X(i, t) which has N×M points, for N genes and M time-points, was modeled by two vectors a(i) and b(i) of size N, and one vector A(t) of size M, for a total of 2*N+M variables. The standard method of least mean squares solution for such bilinear problems employs singular value decomposition (SVD) (34,35). First the mean over i of u(i,t) was removed u(i,t)=u(i,t)−<u(i,t)>. A(t) is the SVD eigenvector with the largest eigenvalue of the matrix

$$J ( t , {t}^{′} ) = {\sum }_{i} \underline{u} ( i , t ) \cdot \underline{u} ( i , {t}^{′} ) .$$

The results for A(t) were normalized to fit the constraints A(t=0)=1 and A(t)>0. A second round of optimization was then performed for β(i) and k(i) using a non-linear least mean squares solver (lsqnonlin, Matlab 5.3) to minimize (X_{measured}-X_{predicted}).

Parameterization Algorithm IV—Error Evaluation:

The quality of the model in describing the data is given by the mean error for each promoter

$${E}_{i} = \frac{1}{NT} {\sum }_{j = 1}^{N} {\sum }_{t = 1}^{T} ( \frac{\vert {X}_{ijt}^{measured} - {X}_{ijt}^{predicted} \vert}{{X}_{ijt}^{measured}} ) .$$

All calculations were performed with Matlab 5.3 (Mathworks Inc.). The error in the estimate the parameters β and k was determined using a standard graphic method (36). Briefly, the form 1/Xᵢ(t)=1/βᵢ+A(t)/(βᵢ kᵢ) was plotted vs. A(t). From the maximal and minimal slopes of the resulting graphs, the error for 1/(βᵢ kᵢ) was determined. From the maximal and minimal intersections of the graph with the y-axis, the error 1/βᵢ was determined.

Results

Promoter activity profiles for the SOS system. Gfp reporter strains were constructed for eight of the SOS operons. The gfp used in this study becomes fluorescent within minutes after transcription (31) and its degradation rate is negligible. The time dependent experimental signal is smooth enough to be differentiated, yielding a direct measure of the promoter activity (rate of mRNA synthesis). The activity of promoter i, Xᵢ, is proportional to the number of gfp molecules produced per unit time per cell,

Xᵢ(t)=(dGᵢ(t)/dt)/ODᵢ(t) (1)

where Gᵢ(t) is gfp fluorescence from the corresponding reporter strain culture and ODᵢ(t) is the optical density.

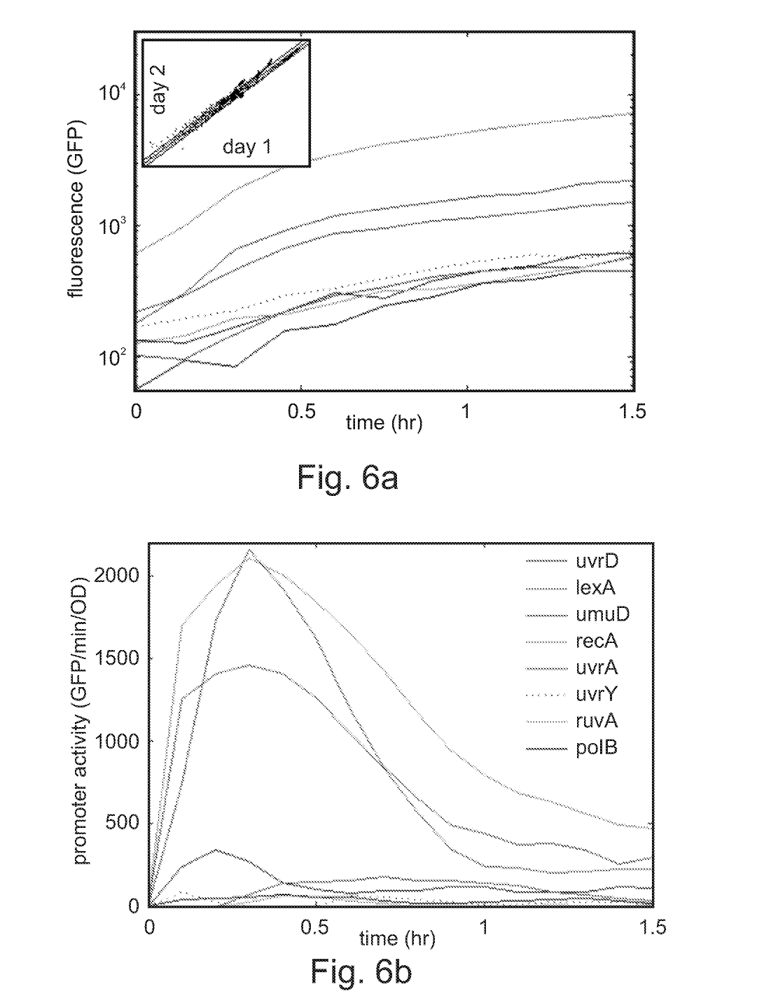

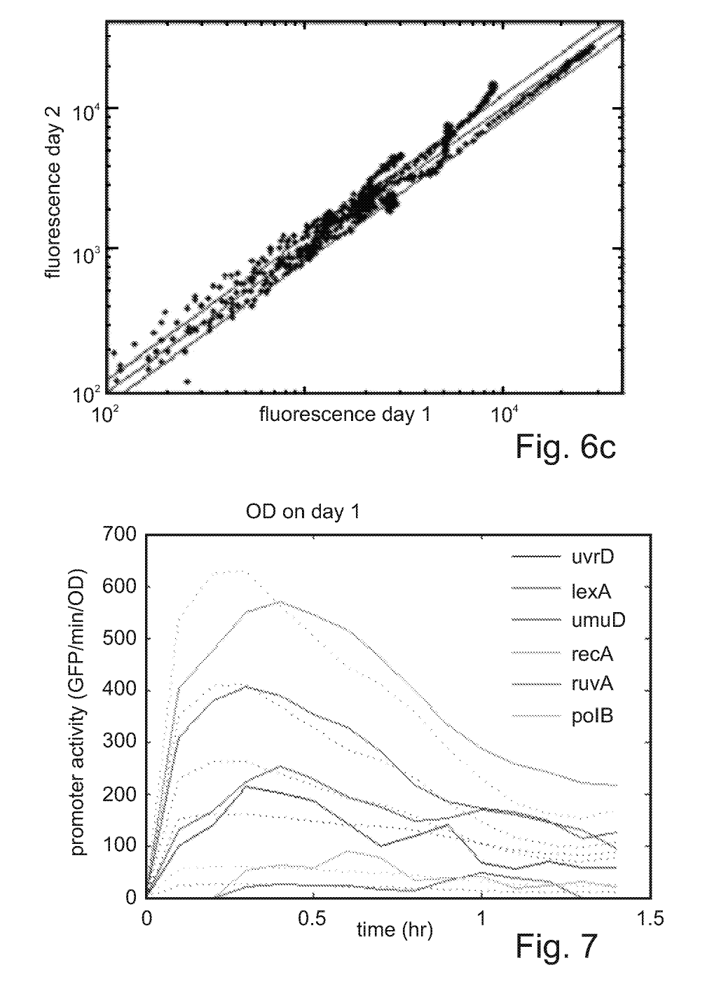

All the SOS operons were activated by UV irradiation (FIG. 6). The time scale for UV induction of the promoters (rise time of ˜7 min) is in agreement with a six time-point DNA microarray experiment. After about half a cell cycle (˜20 min) the promoter activities begin to decrease. This corresponds to the repair of damaged DNA and other adaptation mechanisms (32). The mean reproducibility error between repeat experiments performed on different days is about than 10% (FIG. 6c).

Assigning effective kinetic parameters. The SOS system has a ‘single-input-module’ architecture where a single input transcription factor controls multiple output operons, all with the same regulation sign (repression or activation), and with no additional inputs from other transcription factors (FIG. 5). This is a basic recurring architecture in transcriptional networks, and characterizes over 20 different gene systems in E. coli. An optimization algorithm is employed to parameterize such gene systems, by assigning effective kinetic parameters based on time-course data. A simple Michaelis-Menten model is optionally used for the kinetics:

Xᵢⱼ(t)=βᵢ/(1+Aⱼ(t)/kᵢ) (2)

Where Xᵢⱼ(t) is the activity of promoter i in experiment j, Aⱼ(t) is the effective repressor concentration in experiment j, βᵢ is the production rate of the unrepressed promoter and kᵢ is the effective affinity of the repressor (concentration at half maximal repression). Each kᵢ parameter represents a combination of the binding affinities of the repressor and RNA polymerase for a given promoter, the binding site positions and possibly other factors. An algorithm described in Methods is used to determine the values of βᵢ, kᵢ and A(t) from the data at two UV doses. The error is under 25% for most promoters (Table 1). Other trial functions could be used in place of Eq. 2 (see Methods), and that the results are expected to be insensitive to the mathematical representation used.

Detection of promoters with additional regulation. Promoters that do not belong to the system can be easily detected using this approach because they are assigned a much larger error (eg. 150% error for the lacZ promoter, Table 1). Interestingly, one of the SOS promoters, uvrY, is found to have a large error (˜45%). This operon has been recently found to participate in a signaling system related to stationary phase response (37, 38), and there is evidence that it is regulated by transcription factors other than LexA (39). The relatively large 30% error of polB may perhaps hint that it also has slight, as of yet uncharacterized, additional regulation. In summary, large errors in the present approach may help to detect genes that have additional regulation.

Determining dynamics of entire system based on a single representative. The parameterization procedure produces a quantitative kinetic model of the system dynamical behavior. Once β and k are determined for each operon, one need only measure the kinetics of a single promoter in a new experiment to estimate all other SOS promoter kinetics. The equation for transforming the kinetics of promoter n, Xₙ to that of promoter m, Xₘ is

$${X}_{m} ( t ) = \frac{{β}_{m}}{1 + \frac{{k}_{n}}{{k}_{m}} ( \frac{{β}_{n}}{{X}_{n} ( t )} - 1 )} \qquad ( 3 )$$

The estimated kinetics using data from only one of the operons (uvrA) agree quite well with the measured kinetics for all operons (FIG. 7). The same level of agreement is found using any of the other operons as the representative. Equation 3 depends on the ratios of the kinetic constants. The ratios kₘ/kₙ and βₘ/βₙ were found to be the same in growth in rich (LB) and minimal (M9) media, at 30° C. and 37° C., and in two different E. coli strains, MG1655 and AB1157 (not shown).

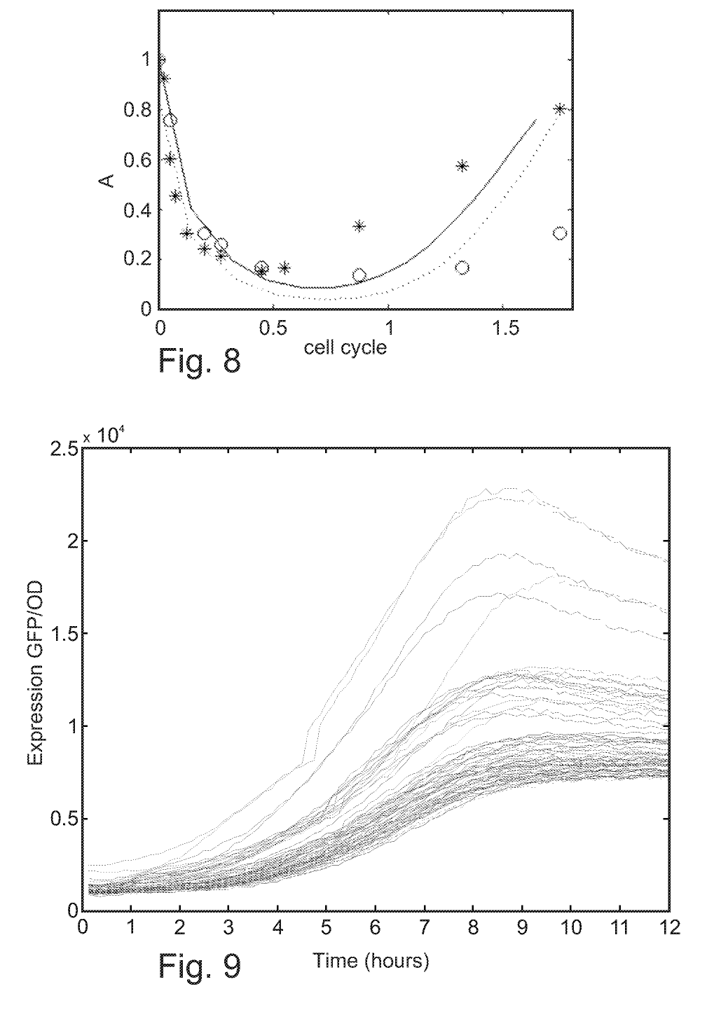

Repressor protein concentration profile. The present measurements are at the transcription level, where GFP is produced under the control of different promoters. The concentrations of the proteins produced by these operons are not directly measured, but only the rate at which the corresponding mRNAs are produced. However, the parameterization algorithm allows calculation of the relative concentration of the master transcriptional repressor (LexA) in its active form using the transcription kinetics (FIG. 8). The calculated concentration, Aⱼ(t), decreases after UV irradiation, reaches a minimum at about half a cell cycle, and then recovers. The predicted relative protein levels are reasonably similar to the immunoblot measurements of LexA protein level in the same strain and conditions reported by Sassanfar and Roberts (32), in particular at early times.

Discussion

The present study demonstrated that effective kinetic parameters could be determined for a transcriptional regulation system of known structure. This was based on algorithms that determine the kinetic parameters within a mathematical model of the regulatory network using accurate promoter-activity measurements.

Detailed temporal program of expression in the SOS DNA repair system. The parameters kᵢ, which qualitatively correspond to the threshold of activation of each operon, are the main parameters that control the kinetics of a ‘single-input-module’ system. In the case of a repressor whose concentration varies with time, the larger the kᵢ value, the earlier the gene is turned on and the later it is turned off. In the SOS system, the initial decrease in LexA levels is very rapid, and thus the operons turn on at about the same time. These operons do turn off, however, at different times, with timing differences on the order of 10 min between operons. The first operons to turn off (smallest k values) are uvrA, part of the earliest repair process, nucleotide excision repair, and lexA and recA, the SOS regulatory genes. Next is umuDC, which encodes for mutagenesis repair enzymes that allows the replication forks to bypass the lesions and resume DNA replication (30, 31). The last genes to turn off are polB, which is involved in replication fork recovery after DNA damage (31), and ruvA and uvrD that are involved in late stage repair processes (uvrD also participates in early repair). The order of inactivation thus correlates with the function of the gene products, with genes responsible for early repair processes turned off first, and those related to recovery and adaptation turned off last. Similar mechanisms may be at play in determining the detailed temporal order in flagella biosynthesis (31) and other systems (32) and may be a recurring motif in transcriptional network dynamics.

Mechanism of SOS system induction. It is generally difficult to measure protein activity profiles in vivo. The present approach addresses this by enabling calculation of the active repressor profile from its transcriptional effects on downstream operons. This compares well with direct immunoblot measurements (FIG. 8). Both the calculated and measured profiles of LexA protein concentration have similar qualitative features. The initial rate of decrease is independent of UV dose (under the present conditions, the cleavage rate is dA/dt≈3 cell-cycle⁻¹), suggesting that the initial cleavage rate of LexA is independent of UV damage. This is consistent with activation of RecA primarily at stalled replication forks. At the UV damage levels used in the present study, there are thousands of lesions in each chromosome, and the replication forks are stalled within seconds after UV irradiation. Since the number of replication forks and the number of RecA monomers activated at each fork are presumably independent of damage level, the initial rate of LexA cleavage is expected to be UV damage independent.

TABLE 1
The effective kinetic parameters for the SOS system (±SD). E is
the mean error for the promoter activity prediction (see Methods).

| | k | β | E | Function |
|---|---|---|---|---|
| uvrA | 0.09 ± 0.04 | 2800 ± 300 | 0.14 | nucleotide excision repair |
| lexA | 0.15 ± 0.08 | 2200 ± 100 | 0.10 | transcriptional repressor |
| recA | 0.16 ± 0.07 | 3300 ± 200 | 0.12 | mediates LexA |
| | | | | autocleavage, blocks |
| | | | | replication forks |
| umuD | 0.19 ± 0.1 | 330 ± 30 | 0.21 | mutagenesis-repair |
| polB | 0.35 ± 0.15 | 70 ± 10 | 0.31 | trans-lesion DNA |
| | | | | synthesis, replication fork |
| | | | | recovery |
| ruvA | 0.37 ± 0.1 | 30 ± 2 | 0.22 | double strand break repair |
| uvrD | 0.65 ± 0.3 | 170 ± 20 | 0.20 | nucleotide excision repair, |
| | | | | recombinational repair |
| uvrY | 0.51 ± 0.25 | 300 ± 200 | 0.45 | SOS operon of unknown |
| | | | | function, additional roles |
| | | | | in two-component |
| | | | | signaling |
| lacZ | — | — | 1.53 | unrelated to SOS system |

### Example 4

### Accuracy of the Present Invention

Current approaches for measuring gene expression are limited in accuracy and in temporal resolution. DNA microarrays measure a snapshot of RNA transcript levels, which therefore require long experiments (spanning 100 time points or more) to be performed with a great deal of labor and many chips. In addition, the accuracy of microarrays is limited due to the need for cell manipulation, which often results in up to 2-fold errors between experiments (ref 41-42).

Although optionally any marker for gene transcription may have been used in order to measure the kinetics of gene expression, promoter activity was chosen as a non-limiting illustrative example of such a marker for the demonstration of the method of the present invention, because reporter plasmids may be used for the determination of promoter activity. GFP (green fluorescent protein) was chosen as the marker gene because this enzyme is fast folding and stable, when inserted to the cells in addition to the LuxCDABE gene, which makes the endogenous substrate for the enzyme and which has low background noise.

### Experimental Methods

The experiments (including analysis of the results) were performed according to the general method of Example 3. Briefly, real-time monitoring of the transcriptional activation of the metabolic pathways was performed with a panel of reporter plasmids in which green fluorescent protein (GFP) is under the control of one of the operon promoters and reporter plasmids in which LuxCDABE is under the control of one of the operon promoters. Particularly, all the plasmids had, in addition to the marker gene, also the gene for kanamycin, and were low copy plasmids of type pCS101.

The reporter plasmids were cloned, 96 at a time, using the polymerase chain reaction, double digestion with two different restriction enzymes, ligation and transformation into bacteria, and then testing the colonies by positive and negative testing methods.

Continuous time courses from living cells grown in a multiwell plate fluorimeter were measured as follows: cultures were grown in a multiwell fluorimeter and assayed with an automatically repeating protocol of fluorescence readings, and absorbance (OD) measurements. Time between repeated measurements was several minutes. 100-300 measurement time points were taken over the 12 hours of growth for the different promoters as shown in FIG. 9. For FIG. 9, every continuous line represents a different promoter for an amino-acid biosynthesis operon of E. coli; it should be noted that since each line is in one of 12 different colors, two different promoters may thus have the same color line.

The high temporal resolution of the present system benefits from the apparent rapid activation of GFP in bacteria as compared with reported times for folding and oxidation of the chromophore in vitro, 10 min and 1 hour, respectively. Due to this advantage, data was received in less than 48 hours even when starting from cells at −80° c.

### Results

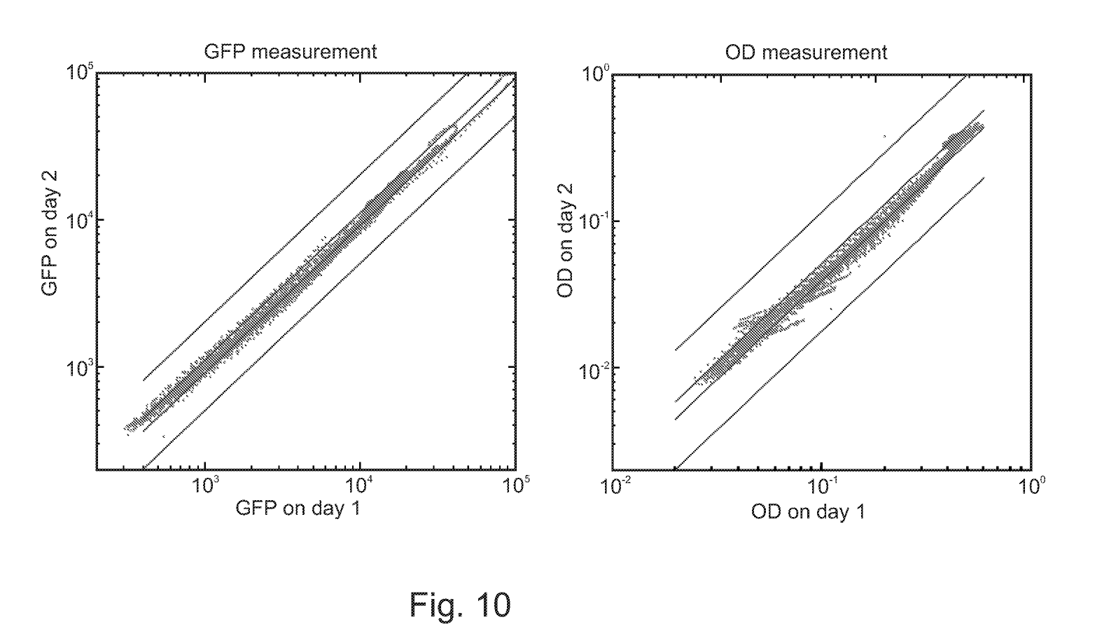

The present experimental method enables continuous time courses from living cells grown in a multiwell plate fluorimeter to be measured. Average errors between repeat experiments were less than 10% (FIG. 10), compared with errors of at least two-fold often associated with expression assays requiring cell lysis and manipulation (refs 10-12).

FIG. 10 shows the high degree of accuracy of the present invention, and the improved accuracy of the present invention over existing technologies such as DNA microarray assays for example.

### Example 5

### Amino Acid Synthesis Pathways

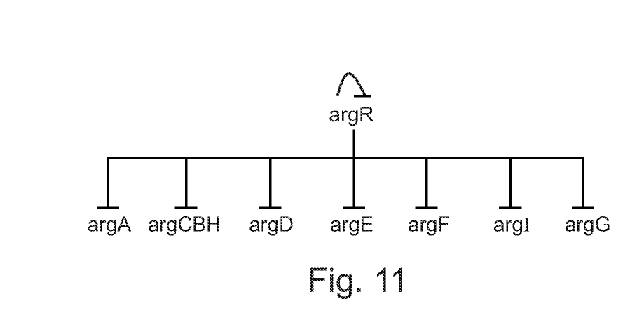

Many amino acid biosynthesis systems are controlled by specific transcription factors. For example, the arginine biosynthesis genes are designed as a single input model (shown as a schematic diagram in FIG. 11). The transcriptional order of gene activity in this pathway is clearly of interest, because a plurality of different genes must be activated in order to obtain the final arginine product. This Example illustrates that the method of the present invention is clearly suitable for determining this transcriptional order.

### Experimental Methods

The experiments (including analysis of the results) were performed according to the general method of Example 3. The same plasmids were used as for Example 4. The main set of experiments was performed on the arginine biosynthetic pathway; however, the cysteine, methionine and serine biosynthetic pathways were also examined, according to similar experimental methods.

For the experiment with the arginine biosynthetic pathway, the reporter strains were inoculated from frozen 96-well plate stocks into minimal medium, supplemented with 0.5% glucose (Sigma), 25 μg/ml kanamycin and 0.05% casamino, both amino acids derived by casein digestion (referred to herein as M9C).

The cultures were grown over night for 16 hours at 37° C. The next day, the cultures were diluted 1:100 into a fresh minimal medium supplemented with 0.5% glucose (M9 medium), 25 μg/ml kanamycin and all the amino acids except arginine. All the dilutions were done in 96 well plates (Nunc) to a total volume of 150 μl. 100 μl of mineral oil (Sigma) was added on top of each well to prevent the evaporation during the automated measurements.

The plates were inserted into an automated Wallac workstation measuring the fluorescence (535 nm), or luminescence and the optical density (600 nm). The parameters of the automated measurements are programmable and are easily defined by the user according to the particular experiment (instructions of the manufacturer were followed for operation). The temperature during the measurements of the expression profile inside the automated machine was 30° C. The interval between two measurements was set to 4 minutes.

As for previous Examples, by measuring the ratio at the same OD, a change in the gene expression in the cells when grown in the two different media can be detected.

The experiments for the cysteine and methionine were performed similarly to those of the arginine study, except that cysteine and methionine were substituted for arginine in the described protocol.

### Results

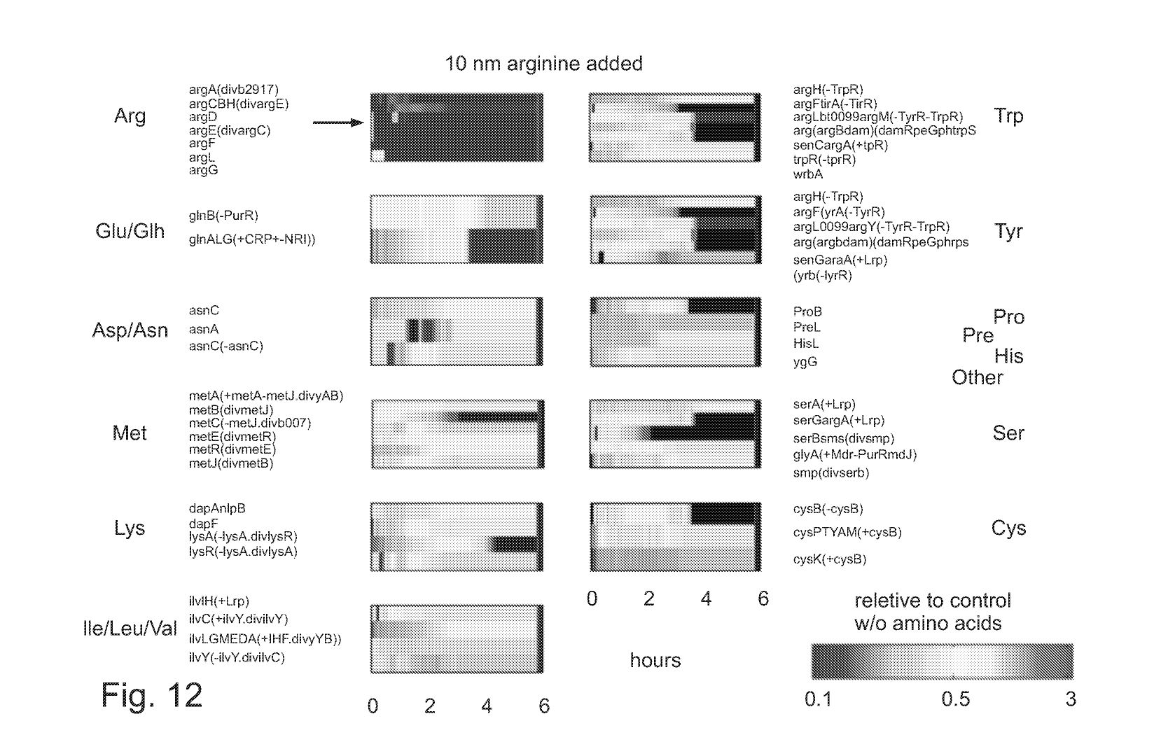

Growing cells in medium which did not contain arginine clearly upregulated gene activity for the genes of the arginine biosynthesis pathway; as expected, the opposite effect was seen for cells grown in arginine-containing medium (shown in FIG. 12).

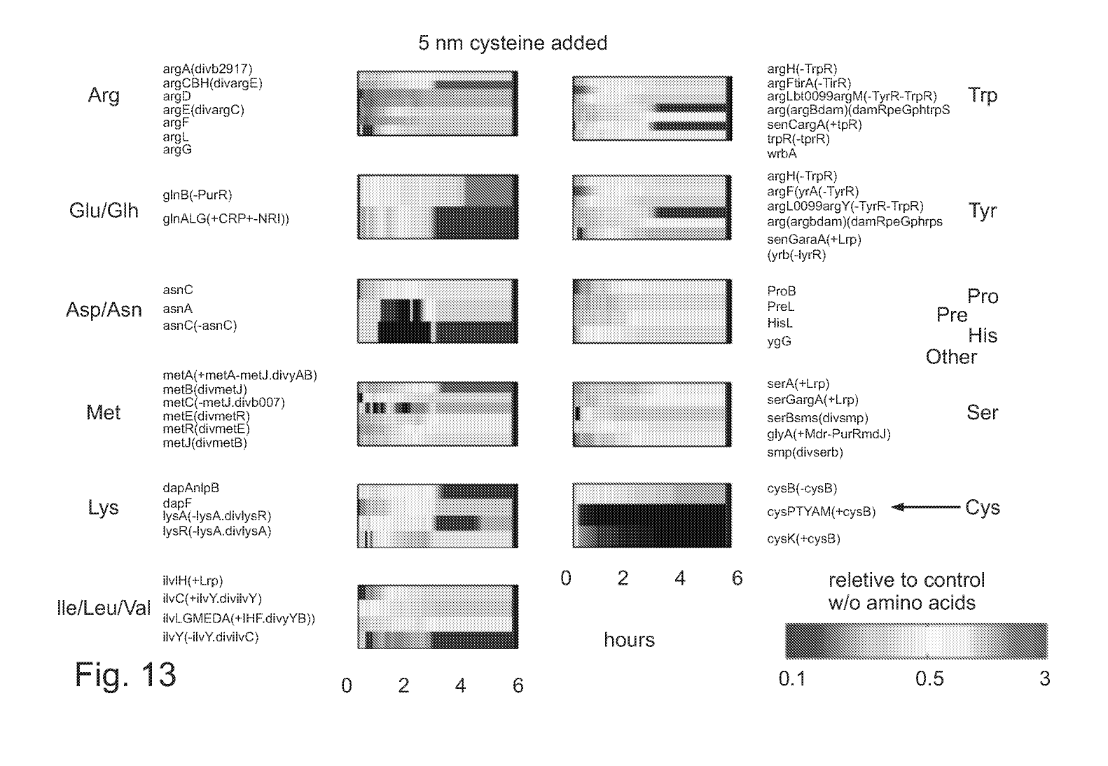

FIG. 13 shows that addition of cysteine leads to down regulation of Cys genes.

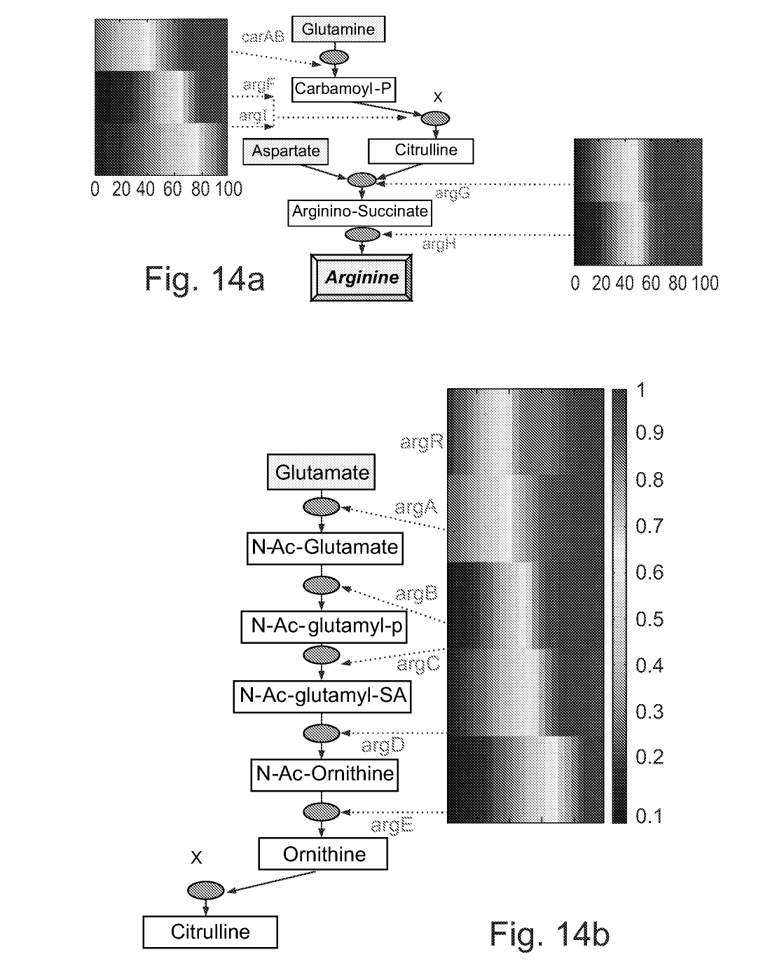

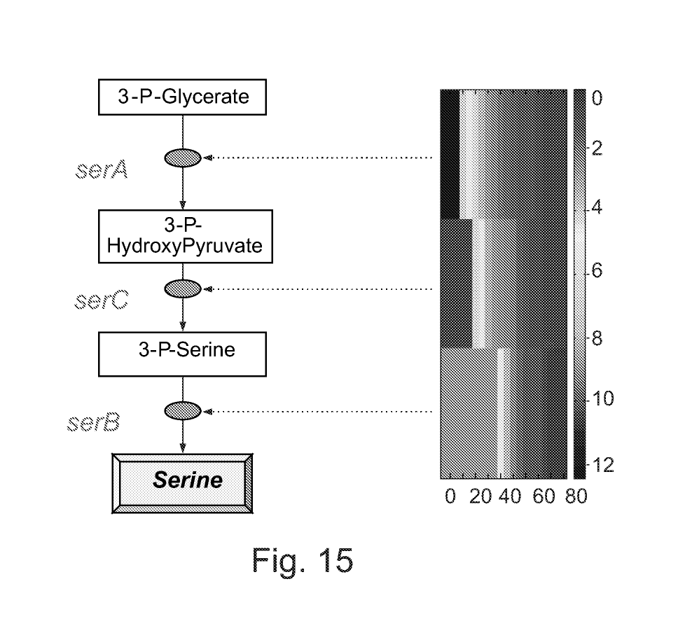

This analysis of the arginine pathway also serves as a specific example of using the method of the present invention for high resolution analysis of such transcriptional activity, with a large number of time points for a higher resolution, as described with regard to the above experimental methods. The results shown in FIGS. 14A and 14B show that the order of gene expression found for the arginine pathway matches the order of the pathway previously described.

FIG. 14A shows argF, argI, argG and argH. FIG. 14B shows argA-E and argR. The connection between the two parts of the pathway is shown at the point “X” (shown as a circle), which is the synthesis leading to citrulline (this part is repeated in both parts of the pathway).

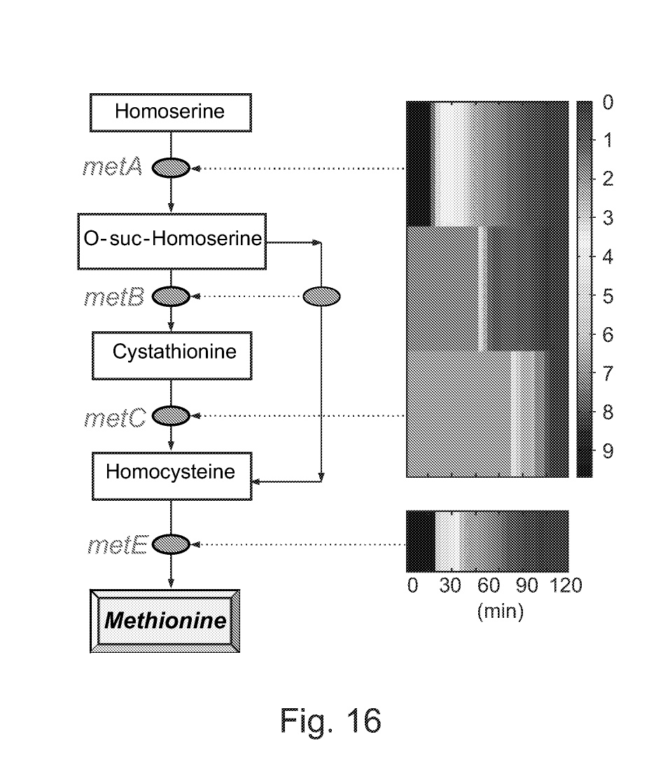

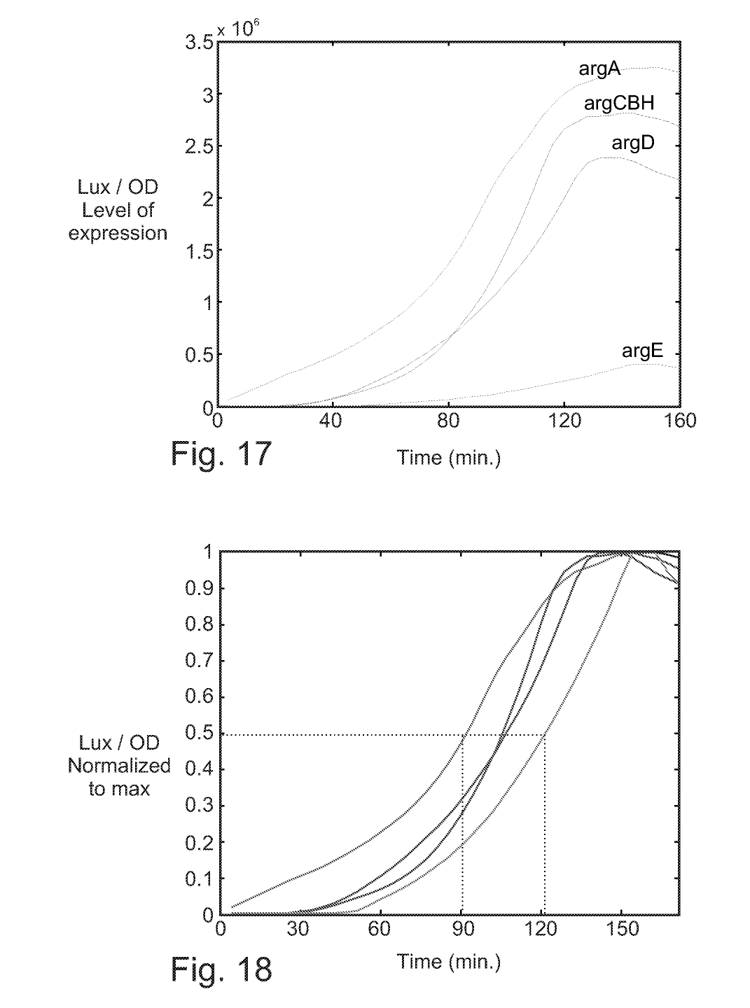

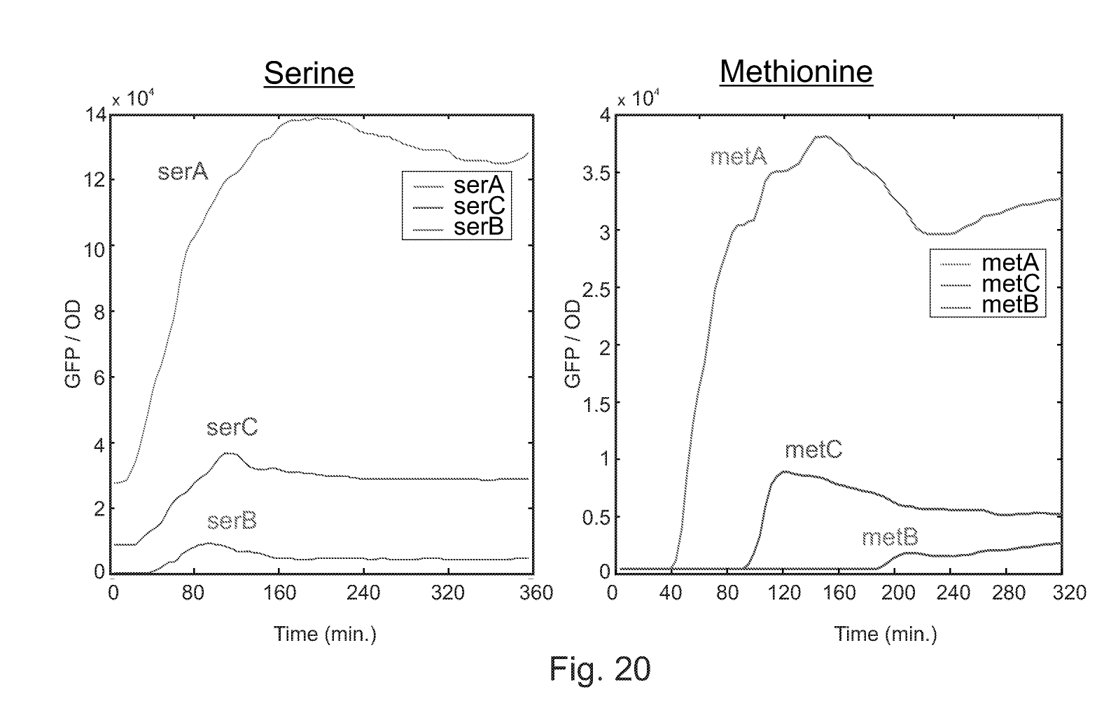

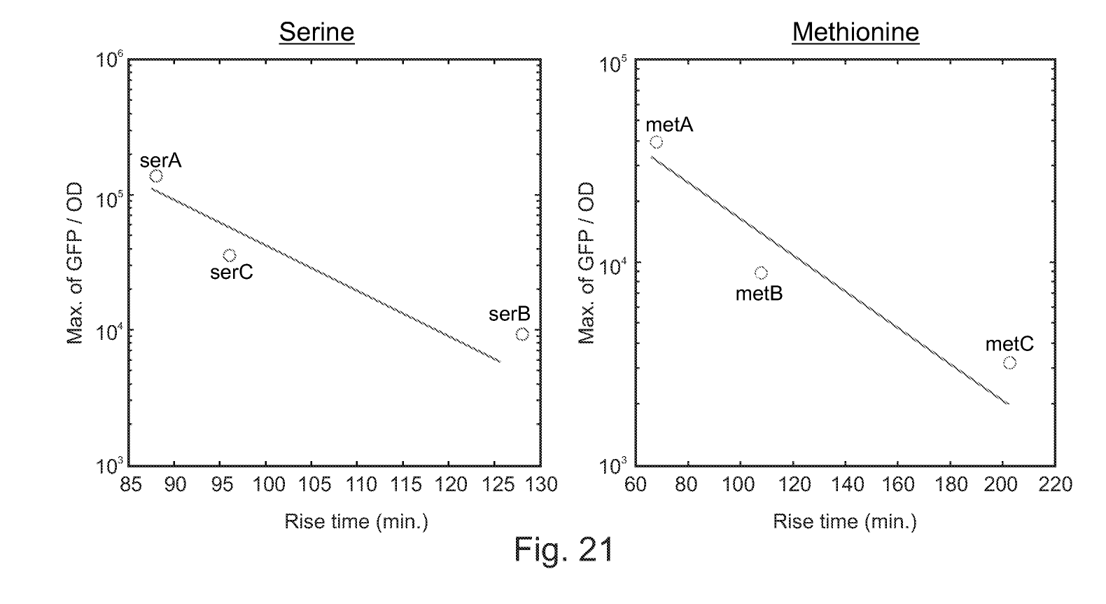

Similar results are also seen for the serine biosynthetic pathway (FIG. 15) and for the methionine biosynthetic pathway (FIG. 16). In addition, it was observed that the earlier the gene appears in the pathway, the higher maximal non-normalized promoter activity it has, as shown with regard to FIG. 17 (x-axis shows level of expression, y-axis shows time; results are shown for expression of the genes argA, argCBH, argD and argE). Similar results are shown for serine (left) and methionine (right) in FIG. 20.

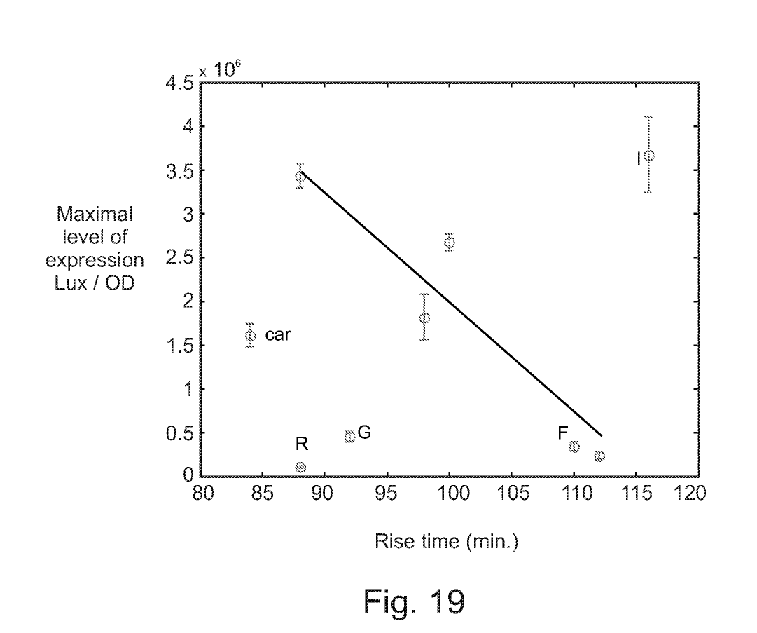

FIG. 18 shows that enzymes involved in early stages of linear pathways have faster rise times visible when checking normalized gene expression (the data shown in FIG. 17 were normalized for this graph). It was also shown that in a pathway, the lower the rise time, the higher the maximal response for the pathway (shown in FIG. 19; similar results are shown for serine (left) and methionine (right) in FIG. 21).

The mechanism for regulation of metabolic pathways can be deduced from the results shown here. Without wishing to be limited to a single hypothesis, the temporal order can be controlled by differential activation coefficients for each promoter for repressor affinity, and the expression hierarchy can be controlled by differential mRNA polymerase binding affinities, which relates to promoter activity. These are parameters that are deducible for each gene in the pathway.

The regulation of genes in pathways ensures that only the genes needed are expressed, only when they are needed, and only at the amount that is needed. This enhances the efficiency of the transcriptional system, and allows for a more rapid response of the system to change.

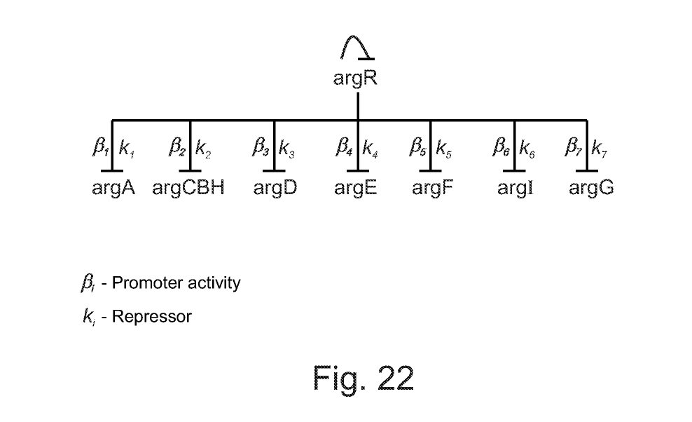

This concept is examined mathematically with regard to FIG. 22, which shows the arginine synthesis pathway again, and the various parameters for promoter and repressor activity. This pathway could be simplified to the following equation:

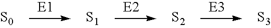

Variables: expression rates of enzymes E1, E2, E3 (for producing products S1, S2 and S3).

### Constraints:

Fixed production rate of the final Product (goal)=what is needed

Minimize total number of enzyme molecules/cell=in the amount needed

Fast response time (time to 50% max production)=quick response to change

From this information, it can be seen that the most efficient biosynthetic pathway would be one which minimizes E1+E2+E3 and the response time in order to produce a given amount of the final product.

### Example 6

### Analysis of a Parasite Life Cycle

Parasites, particularly those which infect humans, are highly problematic for treatment. Elucidation of the temporal order of the expression of the genes of such organisms could help target new treatments according to the form and behavior of the organism at different life stages.

The background art shows that some of the malaria parasite genes, are specific to a certain stage of the parasite life cycle, such as HRP-1 and MSP-1 which are specific to the Trophozoite stage, and Pfs25 which is a gametocyte specific gene (ref 40). It should be noted in the same reference which described these different stage-specific genes, many more genes were found to be stage specific but which had unknown sequences. Lack of knowledge about such genes prevents the function of the resultant proteins from being determined. However, currently available methods (such as those described in ref 40) are clearly not sufficient for determining function. For example, such methods cannot ascertain the temporal order of gene expression relative to other known and/or unknown genes.

The present invention, by contrast, could be used to deduce such a temporal order. Determination of the temporal order of the expression of these genes would thereby enable doctors to treat the infected persons in a way which is most efficient against the parasite at the stage it is being carried. It would also be useful for drug development, including for targeted or directed drug development.

The present invention has a clear advantage over the methods currently available. The full malaria genome is yet to be sequenced fully, and even when sequenced, about half the malarial genome coding regions are expected to have unknown function, making parasitic biology very challenging to study. Moreover, the complete sexual life cycle of the parasite can only be studied in mosquitoes, and not yet in vitro, making it difficult to perform classical genetic experiments on such parasites. Impractical quantities of parasites are needed in order to obtain native proteins in sufficient quantity for microsequencing, creating another aspect of difficulty in research using currently available methods (ref 40).

The method of the present invention can be used to find the temporal network in gene expression of the malaria parasite, solving the problems existing in the background art and supplying an urgent need for additional methods for assessing gene function in malaria.

As a non-limiting, illustrative example, the method of the present invention could optionally be used by measuring gene expression using a marker gene such as the GFP gene as described in the previous Examples, which could easily be determined by one of ordinary skill in the art from the information contained in this application. Furthermore, the kinetics of the gene expression for the different genes tested can also be determined. A non-limiting illustrative example of such genes is the network of genes specific to the trophozoite stage, such as the HRP-1 gene. Next, a quantitative value is determined for each parameter of the kinetics of at least some of the genes, and for at least one of them a gene expression profile is preferably created. Preferably, an extrapolation can be performed to calculate the quantitative value for all the genes involved from the parameters of the kinetics and this expression profile.

By understanding the different quantitative values for each of the genes at the different times which the temporal network spans, (and preferably having a profile of protein concentrations for them) one of ordinary skill in the art may be able to use DNA, RNA transcript and/or protein concentration clinical testing for organisms infected with the parasite, for specific diagnosis and treatment. Optionally and additionally or alternatively, special treatment regimens may be designed to eliminate the parasite at different stages in its life cycle.

### Example 7

### Cancer Diagnosis and Treatment

The background art shows that some genes are expressed differentially between tumor cells and non-cancerous cells, and that there are coherent patterns of genes whose expression is correlated, suggesting a high degree of organization underlying gene expression in the different tissues. It was also shown that the above tissue types can be separated on the basis of the gene expression profile found in them. Similar results were described also across several other tissue types. In addition, recent work demonstrates that genes of related function could be grouped together according to similar temporal evolution under various conditions. For example, in order to understand the p53 signaling network, it is necessary to look not only at isolated components, but at the whole network, with time being an important factor in this understanding, due to the rapid changes the protein and expression of the p53 gene undergo in respond to several feedback and feedforward systems that are part of the stimulus in the network (ref 43-44).

From these previous findings, it is clear that the present invention could optionally and preferably be used for cancer diagnosis and/or treatment, for example in order to learn which genes are expressed in cancerous tumors in different stages (benign vs. malignant, and different levels of malignancy) and/or the temporal pattern of expression within each stage. This analysis could be used to support clinical RNA/protein testing, to identify the stage of the disease, thereby enabling the doctor to apply the best treatment for the specific patient. The method may also be usable for detecting malignant as opposed to benign tumor cells.

Tissue taken from the patient could preferably be manipulated as for the cells in the previous Examples, for assessing the clinical state of a particular patient. In order to further determine the relationship between clinical state and gene expression, preferably tumors of different types (benign vs. malignant, or tumors at different histopathological stages) are tested for gene expression networks as described. Alternatively or additionally, such tests could be performed by using one tissue which can be actively transferred between the different stages, combined with the method of the present invention.

The tissues are preferably cultured in a fluorimeter, and tested using some marker gene or some other way of testing the level of gene expression over a period of time, or preferably the kinetics of gene expression over the same period of time, preferably according to a method with a high temporal resolution which does not affect the biological function by the measuring. The temporal behavior of the different networks that affect the difference in stage and malignancy between the tumors is then determined by using analysis of the gene expression kinetics found.

The different genes involved in the different malignant functions of the tumor cells can then be clustered into groups according to some distance metric found by use of the gene expression kinetics found in the previous section, based on the correlation between the kinetics of the different genes. The groups are then ordered according to the relative order among them, and then the genes in each group are ordered within the group.

According to the temporal pattern found by ordering all the involved genes in the above way, it would be possible to determine which genes are regulators of crucial functions such as passing between malignancy stages, possibly enabling doctors to delay these transitions, and/or could be used for clinical testing. For example, a patient who is diagnosed with some sort of cancer and/or suspected cancer could have the current gene expression profile of the tissue(s) tested, thereby providing a better diagnosis of the stage of malignancy that was reached, and thus allowing for more accurate and appropriate treatment for the patient.

Modifications of the previously described method of the present invention, from the above examples, could optionally be performed according to background art references which describe the importance of gene expression networks and patterns in cancer (ref 43-44).

### Example 8

### Other Uses of the Present Invention

The method of the present invention, optionally and preferably in conjunction with the present experimental method, can be readily applied to gene systems in a broad range of sequenced prokaryotes, as well as to eukaryotic genes with well-defined regulatory regions. For example, GFP was used to monitor gene expression on a large scale in yeast (18). Studies on various systems could establish whether temporal clustering and memory effects can be a general method in mapping assembly cascades and detecting regulatory checkpoints.

The method according to the present invention could in principle apply to any gene system controlled by a single transcription factor, or gene systems controlled by multiple transcription factors provided that the activities of all but one are held constant during the experiment. The present invention could also optionally be used with systems having multiple varying transcriptional inputs, which requires a quantitative understanding of the ‘cis-regulatory-logic’ that combines multiple inputs at each operon. The present accurate kinetic measurements could be performed in principle at a genomic scale using arrays of reporter strains. This raises the possibility of producing kinetic models and understanding the principles of the dynamical behavior of cell-wide regulatory networks by using the method according to the present invention.

Another example of the possible use of another embodiment of the present invention is for providing a method for analyzing the temporal behavior of a network of stocks, the network being associated with a plurality of stocks and bonds. Such analysis could be performed for example by measuring the rise in the different stocks and bonds over a period of time, or different shorter periods of time that have different influential factors. Preferably, rise in stocks is counted in positive numbers, and fall in negative ones, relative to the day the testing started. More preferably, measuring further consists of using a simple method, such as for example counting the number of stocks sold every hour, as a measurement method with a high temporal resolution that leaves the function of the measured stocks unaltered over the testing period.

Subsequently, the relative distance between each pair of different stocks could optionally be determined. The metric used for finding the relative distance between each pair of stocks can optionally, and in a non-limiting fashion be calculated according to the metric d(i,j)=1−corr(i, j).

The resulting stocks are then clustered into groups according to the found distances—by defining a distance threshold for stocks to be in the same group, and subsequently ordering the groups according to the distances between them.

Next, the stocks in each group should be sorted, according to their temporal order of rise in sales.

This process should result in at least one temporal network of the traded stocks. The results can then be crossed with the background information about the different factors that may have affected the transactions of the discussed stocks (such as important political decisions made over the testing time period, or large business transactions or mergers made during this time), enabling a better understanding of the manner in which different factors affect the changes in trade of the different stocks and bonds. Such a deeper understanding may enable stock dealers, brokers and business executives to more easily handle and understand stocks, and also provide more profitable transactions.

Yet another non-limiting example of an application of the present invention could optionally be for agriculture. This application would involve studying the temporal behavior of some desired agricultural process (for example lactation or gene expression in plant or animal growth and development), by clustering the genes according to their expression level over a period of time. Understanding the different stages of such a network, otherwise known as gene-timing, may enable lengthening (or shortening) the time that this network is expressed in the organism, allowing for a better production rate.

The method is preferably implemented on the desirable network (such as lactation in cows, which is used as a non-limiting, illustrative example only) in a similar way to that described for the flagellar network. A marker gene vector is preferably placed under the control of the different lactation promoters and the cells of the relevant tissues (such as the udder tissue, or parts of the milk gland) are grown in vitro and checked for the expression of the different promoters at fixed time distances for a predefined time span.

The distance is then preferably calculated for every pair of genes according to a chosen distance metric such as the one mentioned above. Different genes are then preferably clustered into groups according to a threshold of relatedness, and then the distances will be recalculated according to the average distance between genes in each group. The groups will then be ordered according to their distances, after lowering the threshold and splitting at least one group of genes into a number of smaller groups according to the lowered threshold distance. The genes within each group are then preferably ordered according to their relative order of expression.

Another non-limiting, illustrative option is to measure the gene expression kinetics for the different genes tested above, using a method with high temporal resolution, and which does not affect the biological function during the measurement. Next, a quantitative value is preferably analyzed, in order to detect some regulatory relationships between the different genes, enabling better understanding of the temporal network.

Once the temporal network is understood, the factors that affect the transition between the different stages can be studied, thereby potentially lengthening of desirable stages (such as the lactation stage), resulting in a larger production of milk, or another desirable product.

These studies can optionally be done on tissues and process networks from many different types of organisms, ranging from bacterial (such as testing the production of penicillin and lengthening the time period of the process) to mammal (such as the lactation process described above), and may even be found useful in humans (for issues such as delaying menopause).

### REFERENCES AND NOTES

1. R. Macnab, in Escherichia coli and Salmonella: Cellular and Molecular Biology, F. C. Neidhart, Ed. (American Society for Microbiology, Washington D.C., 1996), pp. 123-145.
2. G. S. Chilcott, K. T. Hughes, Microbiol. Mol. Biol. Rev. 64, 694. (2000).
3. K. Kutsukake, Y. Ohya, T. Iino, J. Bacteriol. 172, 741 (1990).
4. Y. Komeda, J. Bacteriol. 168, 1315 (1986).
5. Y. Komeda, J. Bacteriol. 150, 16 (1982).
6. B. P. Cormack, R. H. Valdivia, S. Falkow, Gene 173, 33 (1996).
7. RP437 (19) and YK410 (20) are E. coli K12 strains that are wild type for motility and chemotaxis. The polymerase chain reaction was used to amplify the flagellar promoter regions using primers designed from the MG1655 genome sequence (21). The promoter region coordinates are flhD (1976454-1976212), flgB (1130044-1130245), flgA (1130245-1130044), fliA (2000123-1999779), fliD (2001594-2001916), fliC (2001916-2001594), fliE (2011261-2010998), fliF (2010998-2011261), fliL (2017491-2017644), meche (1970893-1970676), mocha (1975301-1975161), flgM (1129471-1129331), flgK (1137467-1137656), and flhB (1964392-1964190). Reporter plasmids were constructed by subcloning these promoter regions into a Bam HI site upstream of a promoterless GFP on the low-copy vector pCS21. pCS21 was constructed by replacing the luciferase gene of pZS21-luc (22) with a DNA fragment containing the GFPmut3 (6) gene. Promoter identity was verified by sequencing. There was no observable effect of the plasmids on swimming motility as assayed on soft agar plates [performed as described (23)], suggesting that the system can compensate for the extra promoter copies introduced by these low-copy plasmids. There were no measurable differences in the growth rate of the reporter strains, with the exception of the reporters for meche, mocha, and flgM, which show a somewhat faster growth in culture. The effective delay in activation of these late class 3 reporters would be further enhanced if one takes into account their faster growth
8. B. M. Pruss and P. Matsumura, J. Bacteriol. 179, 5602 (1997).
9. J. E. Karlinsey et al, Mol. Microbiol. 37, 1220 (2000).
10. P. T. Spellman et al., Mol. Biol. Cell 9, 3273 (1998).
11. R. Zhao et al., Genes Dev. 14, 981 (2000).
12. S. Chu et al., Science 282, 699 (1998).
13. M. B. Eisen, P. T. Spellman, P. O. Brown, D. Botstein, Proc. Natl. Acad. Sci. U.S.A. 95, 14863 (1998).
14. Cultures (2 ml) inoculated from single colonies were grown 16 hours in Tryptone broth (Bio 101, Inc.) with kanamycin (25 μg/ml) at 37° C. with shaking at 300 rpm. The cultures were diluted 1:600 or 1:60 into defined medium [M9 minimal salts (Bio 101, Inc.)+0.1 mM CaCl₂+2 mM MgSO₄+0.4% glycerol+0.1% casamino acids+kanamycin], at a final volume of 150 μl per well in flat-bottomed 96-well plates (Sarsteadt 82.1581.001). The cultures were covered by a 100-μl layer of mineral oil (Sigma M-3516) to prevent evaporation during measurement. Cultures were grown in a Wallac Victor2 multiwell fluorimeter set at 30° C. and assayed with an automatically repeating protocol of shaking (1 mm orbital, normal speed, 180 s), fluorescence readings (filters F485, F535, 0.5 s, CW lamp energy 10,000), and absorbance (OD) measurements (600 nm, P600 filter, 0.1 s). Time between repeated measurements was 6 min. Background fluorescence of cells bearing a promotorless GFP vector was subtracted. RP437 was the parental strain of all reporter strains, except flhDC, for which the signal was below background at early time points, and thus YK410 was used. Similar timing and temporal ordering of the flagellar operons was observed in this strain. The high temporal resolution of the present system benefits from the apparent rapid activation of GFP in bacteria (24, 25) as compared with reported times for folding and oxidation of the chromophore in vitro, 10 min and 1 hour, respectively (26).
15. R. O. Duda, P. E. Hart, Pattern Classification and Scene Analysis (Wiley, New York, 1973).
16. D. F. Blair and H. C. Berg, Science 242, 1678 (1988).
17. S. M. Block and H. C. Berg, Nature 309, 470 (1984).
18. D. Dimster-Denk, et al., J. Lipid Res. 40, 850 (1999).
19. J. S. Parkinson and S. E. Houts, J. Bacteriol. 151, 106 (1982).
20. Y. Komeda, K. Kutsukake, T. Iino, Genetics 94, 277 (1980).
21. F. R. Blattner et al., Science 277, 1453 (1997).
22. R. Lutz and H. Bujard, Nucleic Acids Res. 25, 1203 (1997).
23. U. Alon, M. G. Surette, N. Barkai, S. Leibler, Nature 397, 168 (1999).
24. P. Cluzel, M. Surette, S. Leibler, Science 287, 1652 (2000).
25. G. S. Waldo, B. M. Standish, J. Berendzen, T. C. Terwilliger, Nature Biotechnol. 17, 691 (1999).
26. B. G. Reid and G. C. Flynn, Biochemistry 36, 6786 (1997).
27. K. Ohnishi, K. Kutsukake, H. Suzuki, T. Iino, Mol. Microbiol. 6, 3149 (1992).
28. K. T. Hughes, K. L. Gillen, M. J. Semon, J. E. Karlinsey, Science 262, 1277 (1993).
29. C. D. Amsler, M. Cho, P. Matsumura, J. Bacteriol. 175, 6238 (1993).
30. Blattner, F. R., Plunkett, G., 3rd, Bloch, C. A., Perna, N. T., Burland, V., Riley, M., Collado-Vides, J., Glasner, J. D., Rode, C. K., Mayhew, G. F., Gregor, J., Davis, N. W., Kirkpatrick, H. A., Goeden, M. A., Rose, D. J., Mau, B. & Shao, Y. (1997) Science 277, 1453-74.
31. Kalir, S., McClure, J., Pabbaraju, K., Southward, C., Ronen, M., Leibler, S., Surette, M. G. & Alon, U. (2001) Science 292, 2080-3.
32. Sassanfar, M. & Roberts, J. W. (1990) J. Mol. Biol. 212, 79-96.
33. Alon U, Camarena L, Surette M G, Aguera y Arcas B, Liu Y, Leibler S & Stock J B (1998) EMBO J. 17, 4238-48.
34. Press W H, Teukolsky S A, Vetterling W T & Flannery B P (1992) Numerical Recipes in C: The Art of Scientific Computing (Cambridge University Press, Cambridge).
35. Alter O, Brown P O & Botstein D (2000) Proc Natl Acad Sci USA 97, 10101-6.
36. Lichten, W. (1989) American Journal of Physics 57, 1112-1115.
37. Pernestig A K, M. O., Georgellis D. (2001) J Biol Chem 276, 225-31.
38. Mukhopadhyay, S., Audia, J. P., Roy, R. N. & Schellhorn, H. E. (2000) Mol Microbiol 37, 371-81.
39. Wei, Y., Lee, J., Smulski, D. & LaRossa, R. (2001) J Bacteriol 183, 2265-72.
40. Rhian E. Hayward, Joseph L. DeRisi, Suad Alfadhli, David C. Kaslow, Patrick O. Brown & Pradipsinh K. Rathod, (2000) Molec Microbiol, 35: 6-14
41. Khodursky, (2000) PNAS;
42. Oh, (2000) Biothech. Prog.
43. Alon et al., PNAS USA, (1999), vol. 96, pp. 6745-6750
44. Vogelstein et al., Nature, (2000), vol 408, pp. 307-310.

What is claimed is:

1. A method for analyzing the temporal behavior of a plurality of genes for a biological system, comprising:
measuring gene expression for the plurality of genes over a period of time, wherein at least a portion of the plurality of genes are wild type genes; and
determining an order of expression of the plurality of genes.

2. The method of claim 1, wherein said measuring is performed for gene expression in a living cell.

3. The method of claim 1, wherein said measuring comprises measuring a level of gene transcription according to promoter activity for the plurality of genes.

4. The method of claim 1, wherein said determining said order comprises:
determining an expression profile for the plurality of genes; and
comparing said expression profiles.

5. The method of claim 4, wherein said comparing said expression profiles further comprises:
clustering a plurality of genes according to similarity in said expression profiles.

6. The method of claim 1, wherein said measuring said gene expression is performed according to a metric for determining a distance between said genes, said metric being determined according to a correlation of kinetics of said gene expression of each pair of genes.

7. The method of claim 6, wherein said kinetics are measured according to a direct measurement of gene expression.

8. The method of claim 7, wherein said kinetics are measured according to an indirect measurement of a biological activity associated with said gene expression.

9. The method of claim 1, wherein said determining said order of expression further comprises:
grouping the plurality of genes according to relative distances to form a plurality of groups;
ordering said groups of genes according to said relative distances; and
ordering genes within each group according to a temporal order of expression of said genes.

10. The method of claim 9, wherein said grouping of the plurality of genes according to relative distances is performed according to a threshold of relatedness.

11. The method of claim 10, wherein said grouping of the plurality of genes further comprises:
recalculating distances between each pair of groups of genes according to an average distance between genes in each group; and
ordering said groups according to said distances.

12. The method of claim 11, wherein said threshold is lowered and at least one group of genes is split into a plurality of smaller groups according to said lowered threshold of distance.

13. The method of claim 12, wherein said groups of genes are ordered according to a dendogram.

14. The method of claim 9, wherein said ordering said genes within each group is performed by:
determining a relative time of expression of each gene; and
ordering said genes according to said relative times of expression.

15. The method of claim 1, wherein said determining said order of expression further comprises:
determining a quantitative value for a parameter for kinetics of said expression for at least one gene.

16. The method of claim 15, wherein said quantitative value is determined for parameters for the plurality of genes.

17. The method of claim 16, wherein said quantitative values are analyzed to detect at least one regulatory relationship between the plurality of genes.

18. The method of claim 17, wherein said determining said order of expression further comprises:
constructing a mathematical model according to said at least one regulatory relationship and said quantitative values for the plurality of genes.

19. The method of claim 18, further comprising:
determining an optimized mathematical model for the plurality of genes.

20. The method of claim 19, further comprising:
optimizing at least one biological process according to said mathematical model.

21. The method of claim 20, wherein said at least one biological process is related to agriculture.

22. The method of claim 20, wherein said optimizing said at least one biological process is applied for treatment of an animal.

23. The method of claim 22, wherein said optimizing said at least one biological process is applied for treatment of a non-human animal.

24. The method of claim 1, wherein said measuring gene expression further comprises:
constructing a gene construct comprising a promoter for said gene and a marker gene; and
measuring activity of said marker gene according to expression in a cell.

25. The method of claim 24, wherein said marker gene is GFP (green fluorescent protein).

26. The method of claim 1, wherein the biological system comprises a tissue suspected of cancer, the method further comprising:
determining a gene expression profile for said tissue; and
detecting said cancer according to said gene expression profile.

27. The method of claim 26, wherein said detecting further comprises staging said cancer.

28. The method of claim 1, wherein the biological system comprises a parasite, the method further comprising:
determining a gene expression profile for said parasite.

29. The method of claim 28, further comprising:
determining a life cycle stage for said parasite according to said gene expression profile.

30. The method of claim 28, further comprising:
determining a treatment for said parasite according to said gene expression profile.

31. A method for analyzing a biological system, comprising:
measuring gene expression for a plurality of genes over a period of time to determine kinetics of said gene expression according to a measurement method having a high temporal resolution;
selecting at least a subset of genes from said plurality of genes according to said kinetics of said gene expression; and
determining a temporal relationship between genes in said subset of genes for analyzing the biological subsystem, wherein said temporal relationship comprises at least a partial order of gene expression for said plurality of genes.

32. A method for analyzing the temporal behavior of a biological system, the biological system being associated with a plurality of genes, the method comprising:
measuring gene expression for the plurality of genes over a period of time according to a measurement method having a high temporal resolution, wherein a biological function of at least a portion of the plurality of genes is substantially unaltered by said measurement method during said period of time;
determining a relative distance between at least said portion of the plurality of genes according to said measurement method; and
ordering at least said portion of the plurality of genes according to said relative distance.

33. A method for analyzing the temporal behavior of a plurality of genes for a biological system, comprising:
providing a metric for determining a distance between each pair of genes;
measuring gene expression for the plurality of genes over a period of time, wherein said measurement method is capable of measuring said gene expression without substantially altering a biological function of the plurality of genes during said period of time;
grouping the plurality of genes according to relative distances to form a plurality of groups;
ordering said groups of genes according to said relative distances; and
ordering genes within each group according to a temporal order of expression of said genes.

34. The method of claim 33, wherein said metric is determined according to a correlation of kinetics of said gene expression of each pair of genes.

35. The method of claim 34, wherein said kinetics are measured according to a direct measurement of gene expression.

36. The method of claim 35, wherein said kinetics are measured according to an indirect measurement of a biological activity associated with said gene expression.

37. The method of claim 33, wherein said grouping the plurality of genes according to relative distances is performed according to a threshold of relatedness.

38. The method of claim 37, wherein said grouping of the plurality of genes further comprises:
recalculating distances between each pair of groups of genes according to an average distance between genes in each group; and
ordering said groups according to said distances.

39. The method of claim 38, wherein said threshold is lowered and at least one group of genes is split into a plurality of smaller groups according to said lowered threshold of distance.

40. The method of claim 39, wherein said groups of genes are ordered according to a dendogram.

41. The method of claim 34, wherein said ordering said genes within each group is performed by:
determining a relative time of expression of each gene; and
ordering said genes according to said relative times of expression.

42. A method for constructing a mathematical model of expression of a plurality of genes in a biological system, the method comprising:
measuring gene expression for the plurality of genes over a period of time to determine kinetics of said gene expression according to a measurement method having a high temporal resolution;
determining a quantitative value for a parameter for kinetics of said expression for the plurality of genes;
analyzing said quantitative values to detect at least one regulatory relationship between the plurality of genes; and
constructing the mathematical model according to said at least one regulatory relationship and said quantitative values for the plurality of genes.

43. The method of claim 42, wherein said determining said quantitative value further comprises:
determining an expression profile for at least one gene of the plurality of genes; and
extrapolating from said expression profile and said measured gene expression to calculate said quantitative values for the plurality of genes.

44. The method of claim 43, wherein said expression profile further comprises a profile of concentrations of a protein coded by said at least one gene.

45. A method for determining kinetic parameters of a gene regulation system for a plurality of genes, the method comprising:
measuring gene expression for the plurality of genes over a period of time, wherein said measurement method is capable of measuring said gene expression without substantially altering a biological function of the plurality of genes during said period of time; and
analyzing said gene expression according to said measurement method to determine the kinetic parameters.

46. The method of claim 45, further comprising:
constructing the mathematical model according to the kinetic parameters.

47. A method for determining time-dependent regulator activity based on transcriptional activity of a plurality of regulated genes, the genes being regulated through a regulator, the method comprising:
measuring the transcriptional activity for the plurality of genes over a period of time according to a measurement method, said measurement method being capable of measuring the transcriptional activity without substantially altering a biological function of the plurality of genes during said period of time; and
determining time-dependent activity of regulation through the regulator.

48. The method of claim 47, further comprising:
measuring activity of a regulatory protein through said time-dependent activity of regulation through the regulator, wherein said regulatory protein binds to the regulator.

49. A method for detecting an influence of a pattern of a plurality of factors on a plurality of stocks in a stockmarket, the method comprising:
measuring prices for the plurality of stocks over a period of time at a plurality of time points;
determining at least a presence of each of the plurality of factors at each time point; and
determining the pattern according to said at least a presence of the plurality of factors and said prices to detect a potential correlation.
